(19) United States
(12) Patent Application Publication
(10) Pub. No.: US 2011/0110920 A1
(43) Pub. Date: May 12, 2011

(54) METHOD OF TREATING PERIPHERAL ARTERIAL DISEASE

(75) Inventors: Darrell H. Carney, Dickinson, TX (US); Barbara Olszewska-Pazdrak, League City, TX (US); Theresa W. Fossum, College Station, TX (US)

(73) Assignee: OrthoLogic Corp.

(21) Appl. No.: 12/934,366

(22) PCT Filed: Mar. 24, 2009
(86) PCT No.: PCT/US09/01822

§ 371 Date: Jan. 5, 2011

Related U.S. Application Data
(60) Provisional application No. 61/070,878, filed on Mar. 26, 2008.

(51) Int. Cl. A61K 38/48; A61P 9/00

(52) U.S. Cl. 424/94.64

(57) ABSTRACT

An agonist of a non-proteolytically activated thrombin receptor can be used in a method for treating peripheral arterial disease. The agonist can be a thrombin peptide derivative. In some embodiments, the peripheral arterial disease is characterized by intermittent claudication. The thrombin peptide derivatives to be used in the methods can have amino acid sequences similar to a region of thrombin. Usually, the thrombin peptide derivatives are 12-23 amino acid residues in length. In some cases, the thrombin peptide derivatives are dimers, and in particular, dimers that result from formation of a disulfide bond between two cysteine residues of peptide monomers.

### RELATED APPLICATION

This application claims the benefit of U.S. Provisional Application No. 61/070,878, filed on Mar. 26, 2008. The entire teachings of the above application are incorporated herein by reference.

### BACKGROUND OF THE INVENTION

It is estimated that about 10 million people in the United States have peripheral arterial disease (also called “peripheral artery disease” or “PAD”). Approximately one half of patients with PAD also have significant coronary artery disease. PAD affects 12-20 percent of people in the U.S. age 65 and older.

In some patients, symptoms include pain with exertion (intermittent claudication) and poor wound healing. The consequences of PAD can be severe, and include restricted mobility due to pain, amputation, and increased risk of myocardial infarction and stroke. Further options are needed to treat PAD.

### SUMMARY OF THE INVENTION

The invention is a method for treating peripheral arterial disease in a subject in need of treatment, by administering a therapeutically effective amount of an agonist of a non-proteolytically activated thrombin receptor (NPAR agonist), which can be a thrombin peptide derivative. The peripheral arterial disease can be occlusive or functional. In some embodiments, the peripheral arterial disease is characterized by intermittent claudication.

The NPAR agonists can also be used in similar methods to treat hemorrhagic stroke, ischemic stroke, or deep vein thrombosis.

The thrombin peptide derivatives to be used in the methods comprise amino acid sequences identical to, or similar to, a region of thrombin. Usually the thrombin peptide derivatives are 12-23 amino acid residues in length. In some cases, the thrombin peptide derivatives are dimers, and in particular, dimers that result from formation of a disulfide bond between two cysteine residues of peptide monomers.

NPAR agonists may exert their effect by inhibiting apoptosis, and/or by reducing endothelial dysfunction or its effects.

### BRIEF DESCRIPTION OF THE DRAWINGS

FIG. 1 shows X-ray films from a representative western blot and a corresponding bar graph of ratios of densitometry readings from the bands on western blots from experiments examining the effect of TP508 on expression levels of arginase 1 in cells cultured with or without TNFα. Human coronary artery endothelial (HCAE) cells were incubated in the absence (CTR) or presence of TP508 (TP) for 1 h prior to treatment with TNFα for 24 h. Cell lysates were analyzed for human arginase 1 expression by immunoblotting using antibody specific for this enzyme. After stripping, the membrane was reprobed with GAPDH antibody to show protein loading. (Glyceraldehyde-3-phosphate dehydrogenase (GAPDH) levels are unaffected by TNFα or hypoxia.) Corresponding densitometric data represent the mean±SD of 4 independent experiments [p<0.05; CTR vs. TNFα (*) or TNFα vs. TP+TNFα (^{#})].

FIG. 2 shows X-ray films from western blot experiments and a corresponding bar graph of ratios of densitometry readings from the bands on the western blot examining the dose dependence of the effects of TP508 on arginase 1 upregulation by TNFα. HCAE cells were pretreated, with TP508 at the indicated concentration for 1 h before stimulation with TNFα for 24 h and cell lysates were analyzed for arginase 1 expression by immunoblotting. Densitometric analysis shows relative intensities of arginase 1 expression.

FIG. 3 shows X-ray films from western blots and corresponding bar graphs of ratios of densitometry readings from the bands on the western blots from experiments showing the effect of TP508 on expression and phosphorylation (activation) of eNOS. HCAE cells were stimulated with TP508 or TNFα and incubated in normoxic or 1% oxygen (hypoxic) conditions for 24 h. Cell lysates were analyzed for eNOS activation by immunoblotting using antibody specific for eNOS phosphorylated at S1177. After stripping, the membrane was reprobed with antibodies for total eNOS and GAPDH. Corresponding densitometric data represent the mean±SD of 3 independent experiments [*signifies p<0.05; CTR vs. TP(*) (normoxia); CTR vs. TP(*) (hypoxia)].

FIG. 4 shows X-ray films from western blot experiments and corresponding bar graphs of ratios of densitometry readings from the bands on the western blots from experiments examining the dose dependence of the effects of TP508 on eNOS activation. HCAE cells were treated with TP508 at the indicated concentrations for 24 h and analyzed for eNOS activity and expression as in FIG. 3. Densitometric analysis shows changes in eNOS phosphorylation relative to total eNOS after TP508 treatment.

FIG. 5 shows stained gels of products of RT-PCR from experiments designed to investigate the effect of TP508 on eNOS mRNA. Changes in eNOS mRNA expression in cells stimulated with TNFα or TP508 for 6 h were analyzed using RT-PCR.

FIG. 6 is a bar graph of data from quantitative analysis of eNOS mRNA expression. HCAE cells were stimulated with TP508 or TNFα, and incubated in normoxic or hypoxic (1% oxygen) conditions for 24 h (p<0.05 when compared with absence of TP508 (CTR) for each condition). RNA was analyzed for eNOS expression using SYBR Green real-time PCR.

FIG. 7 depicts the encoded amino acid sequence of human pro-thrombin (SEQ ID NO:12). Amino acids 508-530, which contain the thrombin receptor binding domain, are underlined. Thrombin consists of the C-terminal 579 amino acid residues of prothrombin. See GenBank Accession No. AJ972449.

FIG. 8 is a graph showing densitometric analysis of a Western blot of activated endothelial nitric oxide synthase (eNOS) in HCAE cells following treatments with TP508, VEGF or a combination thereof

FIG. 9A is a diagram showing the experimental apparatus and design of experiments to measure migration of endothelial cells toward a chemoattractant.

FIG. 9B is a bar graph showing the effect of TP508 treatment on migration of endothelial cells toward the angiogenic factor VEGF.

FIG. 10A is a diagram showing the experimental apparatus and design of experiments to measure invasion of endothelial cells through Matrigel toward a chemoattractant.

FIG. 10B is a bar graph showing the effect of TP508 treatment on invasion of endothelial cells toward the angiogenic factor VEGF.

FIG. 11 is a graph showing the increase in VEGF mRNA in human cardiac microvascular endothelial cells treated with VEGF or with TP508 under normal oxygen conditions or hypoxic conditions.

FIG. 12 is a graph of data from measurements on rings of rat aorta to assess the relaxant effect of TP508.

FIGS. 13A-13D are graphs showing the extent of dilation of coronary arterioles from ischemic hearts under conditions as shown. FIGS. 13A-13B show endothelium-dependent NO-mediated vasodilation. The degree of endothelial dysfunction was evaluated in arterioles isolated from normoxic control left anterior descending artery (Nonischemic) or ischemic left circumflex coronary artery of placebo (Ischemic placebo) or TP508-treated (Ischemic TP508) hearts by determining the degree of vasodilation as a percentage of maximal dilation in response to indicated concentrations of adenosine (FIG. 13A) or serotonin (FIG. 13B) (*p<0.05 compared with nonischemic; #p<0.05 compared with ischemic placebo). FIGS. 13C and 13D show endothelium-independent NO-mediated vasodilation. To determine potential effects of ischemia and TP508 on NO-independent vasodilation, isolated arterioles were stimulated with the indicated concentrations of nitroprusside (FIG. 13C) or pinacidil (FIG. 13D).

FIG. 13E is a bar graph showing results of treating arterioles isolated from nonischemic and ischemic hearts with either placebo or TP508, and analyzing for NO production.

FIG. 14 is a bar graph showing the extent of the cytotoxic effect of 100 mM H₂O₂ on human umbilical vein endothelial cells treated or not treated with TP508.

FIG. 15A and FIG. 15B are graphs of Annexin V and 7-AAD (7-amino-actinomycin D) positive cells (indicating apoptosis), in mouse fibroblasts grown in serum-free medium and treated with either TP508 or peptide GRGDSP (FIG. 15A) or peptide cRGD (FIG. 15B), followed by H₂O₂ treatment.

FIG. 16 is a bar graph representing the significance of the differences between levels of gene expression in hypoxic (1% O₂) neonatal human microvascular endothelial cells (HMVECs) in the presence versus in the absence of added TP508, for genes in categories A-F. The numbers on the bars represent the number of input genes on these functional process and canonical pathways. (Genes expressed due to hypoxic condition t test p-value <=0.05.) (p<0.05; 999 genes; n=4)

FIG. 17 is a bar graph representing the significance of the differences between levels of gene expression in hypoxic (1% O₂) neonatal human microvascular endothelial cells (HMVECs) in the presence versus in the absence of added TP508, for genes in categories A-K. The numbers on the bars represent the number of input genes on these functional process and canonical pathways. (Genes expressed due to hypoxic condition t test p-value <=0.10.) (p<0.01; 82 genes; n=4)

FIG. 18 is a bar graph representing the significance of the differences between levels of gene expression in adult human microvascular endothelial cells (HMVECs) in the presence versus in the absence of added TP508, for genes in categories A-J. The numbers on the bars represent the number of input genes on these functional process and canonical pathways. (Genes expressed due to hypoxic condition t test p-value <=0.05.) (p<0.05; 418 genes)

### DETAILED DESCRIPTION OF THE INVENTION

Peripheral arterial disease is a subset of peripheral vascular disease. Peripheral arterial disease or peripheral artery disease can occur in arteries other than those supplying blood to the heart, but most often occurs in the legs and feet. The disease is characterized by segmental lesions causing stenosis or occlusion, usually in large and medium-sized arteries. Atherosclerosis is the leading cause of PAD, which results in atherosclerotic plaques with calcium deposition, thinning of the media, patchy destruction of muscle and elastic fibers, fragmentation of the internal elastic lamina, and thrombi composed of platelets and fibrin.

Common sites for PAD are the femoral and popliteal arteries, (80 to 90% of patients), the abdominal aorta and iliac arteries (30% of patients) and the distal vessels, including the tibial artery and peroneal artery (40-50% of patients). The incidence of distal lesions increases with diabetes and with age.

PAD can be diagnosed by weak or absent pulses distal to the obstruction. The ankle-brachial index test (ABI) is performed to compare blood pressure in the ankles to blood pressure in the arms. The ratio of ankle to brachial systolic pressure is normally at least 0.9, and by some studies, is at least 1.0. A ratio of less than 0.5 is an indication of severe PAD. A ratio of 0.5 to 0.7 is an indication of moderate PAD. Other diagnostic tests that can be used are Doppler and ultrasound imaging to measure blood flow, computed tomographic angiography (CT), magnetic resonance angiography (MRA) and angiography.

Available treatments include lifestyle changes, among them, smoking cessation, increased exercise, and dietary alterations to decrease cholesterol and fat. Medications are often prescribed to lower blood pressure, help prevent blood clots, and decrease buildup of plaque in the arteries. In other cases, PAD is treated with percutaneous transluminal angioplasty (balloon angioplasty) with or without placement of a stent, atherectomy, in which the blocked region of the artery is “shaved” away by a tiny device on the end of a catheter, or by laser angioplasty, in which a laser is used to remove the blockage in the artery. Surgical options are also available, in more severe cases. Bypass surgery can use saphenous vein bypass grafts or synthetic grafts to reroute blood flow. Any of these available treatments can be combined with a method of treatment disclosed herein.

Half of the patients found to have PAD do not experience symptoms. The most common symptom of PAD is intermittent claudication, leg pain that occurs when walking or performing other exercise, and disappears with cessation of the activity. There may also be pain at rest, numbness, tingling in the lower legs and feet, cold legs and feet, and ulcers on the legs and feet that do not heal or are slow to heal.

Conditions associated with PAD may be occlusive or functional. Examples of occlusive PAD include peripheral arterial occlusion, which may be acute, and Buerger's disease (thomboangiitis obliterans). Examples of functional PAD include Raynaud's disease, Raynaud's phenomenon and acrocyanosis.

Acute peripheral arterial occlusion results in the sudden cessation of blood flow to an extremity of the body, either due to the local accumulation of cells and material within the artery (thrombus) or because a clot has been brought to its site of lodgement by the blood current (embolism). Typically, the affected limb becomes pale, feels cold and becomes painful.

Atheroembolism, a subset of acute arterial occlusion, occurs when multiple small deposits of debris embolize from proximal atherosclerotic lesions and lodge in the small vessels of the muscle and skin. The site of embolization may be acutely painful. Necrosis and gangrene may develop in a digit.

Buerger's disease (also called thromboangiitis obliterans) is a chronic inflammation of the arteries and veins in the arms and legs. Eventually, blood clots form in the blood vessels and block blood flow in and out of the hands and feet. Symptoms include pain, numbness, tingling in the fingers and toes, which can progress to proximal parts of the limbs. Wounds to the hands and/or feet may be slow to heal. Cold sensitivity may develop in the hands and feet. Radial, ulnar, and/or tibial pulses are weak or absent. Proximal athrosclerotic disease is usually absent. Almost everyone diagnosed with Buerger's disease smokes cigarettes or uses tobacco in another form. The damage from Buerger's disease can lead to gangrene in fingers or toes, requiring amputation. The cessation of smoking can stop the progression of the disease.

Another subset of peripheral vascular disease includes thrombophlebitis, which is a condition in which a blood clot forms in a vein. If the vein is near the surface of the body, it is superficial thrombophlebitis. If the vein is deep within a muscle, the condition is deep vein thrombosis.

Superficial thrombophlebitis can occur, for example, following a minor injury to a vein. Symptoms may include swelling, redness and warmth in the area around the vein. The vein may be palpable as a hard and tender cord under the surface of the skin. Serious complications are rare; the condition usually subsides within a week or two.

The risk of deep vein thrombosis is increased by major surgery, injury, increased estrogen, heart disease, cancer, advanced age, obesity, underlying clotting disorders, smoking, and conditions causing long periods of immobility. Symptoms may include skin redness, pain or swelling in the part of the body affected, most often the leg, and warmth and tenderness over the vein. Complications may include pulmonary embolism, and less frequently, heart attack or stroke.

Treatment of deep vein thrombosis usually includes administration of an anticoagulant. Other measures may be taken to prevent recurrence of deep vein thrombosis.

Hemorrhagic stroke or cerebral hemorrhage can occur as a result of trauma, ruptured aneurysm, hypertension or arteriovenous malformation. Neurological symptoms result from the toxic effect of blood.

Ischemic stroke can occur as a result of thrombosis or embolism. Risk factors include, for example, advanced age, family history of ischemic stroke, hypertension, diabetes, and atrial fibrillation.

Compounds which stimulate a non-proteolytically activated thrombin receptor (NPAR) are said to be NPAR agonists. One such NPAR is a high-affinity thrombin receptor present on the surface of most cells. This NPAR component is largely responsible for high-affinity binding of thrombin, proteolytically inactivated thrombin, and thrombin derived peptides to cells. This NPAR appears to mediate a number of cellular signals that are initiated by thrombin independent of its proteolytic activity (see Sower, et. al., Experimental Cell Research 247:422 (1999)). This NPAR is therefore characterized by its high affinity interaction with thrombin at cell surfaces and its activation by proteolytically inactive derivatives of thrombin and thrombin derived peptide agonists as described below. NPAR activation can be assayed based on the ability of molecules to stimulate cell proliferation when added to fibroblasts in the presence of submitogenic concentrations of thrombin or molecules that activate protein kinase C, as disclosed in U.S. Pat. Nos. 5,352,664 and 5,500,412. The entire teachings of these patents are incorporated herein by reference. NPAR agonists can be identified by this activation or by their ability to compete with ¹²⁵I-thrombin binding to cells.

One or more NPAR agonists, and in particular, one or more thrombin peptide derivatives, can be used in methods to treat peripheral arterial disease, thrombophlebitis, or to treat hemorrhagic stroke or ischemic stroke. Compositions comprising NPAR agonists can be administered to a subject in need of treatment of the conditions described herein. Treatment can ameliorate the condition or alleviate the symptoms thereof. NPAR agonists can be administered to subjects who can benefit from therapeutic intervention causing complete or partial alleviation of symptoms. NPAR agonists can be administered to subjects, (e.g., human patients) at risk for developing a disorder described herein, to reduce the probability of developing the disorder. For example, treatment can cause a reduction in the probability of developing the disorder by up to 20, 30, 40, 50, 60, 70, 80, or 90 percent. Treatment can in some cases, delay the development of a disorder, reduce symptoms, or delay severity of symptoms.

Additional embodiments of the invention relate to the administration of a thrombin peptide derivative, for example, as part of a pharmaceutical composition, comprising a pharmaceutically acceptable carrier, to achieve any of the physiological effects discussed herein.

A thrombin receptor binding domain is defined as a polypeptide or portion of a polypeptide which directly binds to the thrombin receptor and/or competitively inhibits binding between high-affinity thrombin receptors and alpha-thrombin. In one embodiment, the thrombin receptor binding domain or portion thereof comprises the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). Another portion of a thrombin receptor binding domain comprises the amino acid sequence Glu-Gly-Lys-Arg-Gly-Asp-Ala-Cys-Glu-Gly (SEQ ID NO:7).

NPAR agonists of the present invention include thrombin peptide derivatives, modified thrombin peptide derivatives and thrombin peptide derivative dimers as disclosed herein.

### Thrombin Peptide Derivatives

Among NPAR agonists are thrombin peptide derivatives, which are analogs of thrombin that have an amino acid sequence derived at least in part from that of thrombin and are active at a non-proteolytically activated thrombin receptor. Thrombin peptide derivatives include, for example, peptides that are produced by recombinant DNA methods, peptides produced by enzymatic digestion of thrombin, and peptides produced synthetically, which can comprise amino acid substitutions compared to thrombin, and/or modified amino acid residues, especially at the termini.

NPAR agonists of the present invention include thrombin derivative peptides described in U.S. Pat. Nos. 5,352,664 and 5,500,412. In one embodiment, the NPAR agonist of the present invention is a thrombin peptide derivative or a physiologically functional equivalent, i.e., a polypeptide with no more than about fifty amino acid residues, preferably no more than about thirty amino acid residues and having sufficient homology to the fragment of human thrombin corresponding to thrombin amino acid residues 508-530 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val; SEQ ID NO:6) that the polypeptide activates NPAR. The thrombin peptide derivatives or modified thrombin peptide derivatives described herein preferably have from about 12 to about 23 amino acid residues, more preferably from about 19 to about 23 amino acid residues.

In another embodiment, the NPAR agonist of the present invention is a thrombin peptide derivative comprising a moiety represented by Structural Formula (I):

Asp-Ala-R (I).

R is a serine esterase conserved domain; Serine esterases (e.g., trypsin, thrombin, chymotrypsin and the like) have a region that is highly conserved. “Serine esterase conserved domain” refers to a polypeptide having the amino acid sequence of one of these conserved regions or is sufficiently homologous to one of these conserved regions such that the thrombin peptide derivative retains NPAR activating ability.

A physiologically functional equivalent of a thrombin derivative encompasses molecules which differ from thrombin derivatives in particulars which do not affect the function of the thrombin receptor binding domain or the serine esterase conserved amino acid sequence. Such particulars may include, but are not limited to, conservative amino acid substitutions and modifications, for example, amidation of the carboxyl terminus, acetylation of the amino terminus, conjugation of the polypeptide to a physiologically inert carrier molecule, or sequence alterations in accordance with the serine esterase conserved sequences.

A domain having a serine esterase conserved sequence can comprise a polypeptide sequence containing 4-12 of the N-terminal amino acid residues of the dodecapeptide previously shown to be highly conserved among serine proteases (Asp-X₁-Cys-X₂-Gly-Asp-Ser-Gly-Gly-Pro-X₃-Val; SEQ ID NO:13); wherein X₁ is either Ala or Ser; X₂ is either Glu or Gln; and X₃ is Phe, Met, Leu, His, or Val.

In one embodiment, the serine esterase conserved sequence comprises the amino acid sequence Cys-Glu-Gly-Asp-Ser-Gly-Gly-Pro-Phe-Val (SEQ ID NO:14) or a C-terminal truncated fragment of a polypeptide having the amino acid sequence of SEQ ID NO:14. It is understood, however, that zero, one, two or three amino acid residues in the serine esterase conserved sequence can differ from the corresponding amino acid in SEQ ID NO:14. Preferably, the amino acid residues in the serine esterase conserved sequence which differ from the corresponding amino acid in SEQ ID NO:14 are conservative substitutions, and are more preferably highly conservative substitutions. A “C-terminal truncated fragment” refers to a fragment remaining after removing an amino acid residue or block of amino acid residues from the C-terminus, said fragment having at least six and more preferably at least nine amino acid residues.

In another embodiment, the serine esterase conserved sequence comprises the amino acid sequence of SEQ ID NO:15 (Cys-X₁-Gly-Asp-Ser-Gly-Gly-Pro-X₂-Val; X₁ is Glu or Gln and X₂ is Phe, Met, Leu, His or Val) or a C-terminal truncated fragment thereof having at least six amino acid residues, preferably at least nine amino acid residues.

In a preferred embodiment, the thrombin peptide derivative comprises a serine esterase conserved sequence and a polypeptide having a more specific thrombin amino acid sequence Arg-Gly-Asp-Ala (SEQ ID NO:16). One example of a thrombin peptide derivative of this type comprises Arg-Gly-Asp-Ala-Cys-X₁-Gly-Asp-Ser-Gly-Gly-Pro-X₂-Val (SEQ ID NO:1). X₁ and X₂ are as defined above. The thrombin peptide derivative can comprise the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6), or an N-terminal truncated fragment thereof, provided that zero, one, two or three amino acid residues at positions 1-9 in the thrombin peptide derivative differ from the amino acid residue at the corresponding position of SEQ ID NO:6. Preferably, the amino acid residues in the thrombin peptide derivative which differ from the corresponding amino acid residues in SEQ ID NO:6 are conservative substitutions, and are more preferably highly conservative substitutions. An “N-terminal truncated fragment” refers to a fragment remaining after removing an amino acid residue or block of amino acid residues from the N-terminus, preferably a block of no more than six amino acid residues, more preferably a block of no more than three amino acid residues.

Optionally, the thrombin peptide derivatives described herein can be amidated at the C-terminus and/or acylated at the N-terminus. In a specific embodiment, the thrombin peptide derivatives comprise a C-terminal amide and optionally comprise an acylated N-terminus, wherein said C-terminal amide is represented by —C(O)NRₐR_{b}, wherein Rₐ and R_{b} are independently hydrogen, a substituted or unsubstituted aliphatic group comprising up to 10 carbon atoms, or Rₐ and R_{b}, taken together with the nitrogen to which they are bonded, form a C₃-C₁₀ non-aromatic heterocyclic group, and said N-terminal acyl group is represented by R_{c}C(O)—, wherein & is hydrogen, a substituted or unsubstituted aliphatic group comprising up to 10 carbon atoms, or a C₃-C₁₀ substituted or unsubstituted aromatic group. In another specific embodiment, the N-terminus of the thrombin peptide derivative is free (i.e., unsubstituted) and the C-terminus is free (i.e., unsubstituted) or amidated, preferably as a carboxamide (i.e., —C(O)NH₂). In a specific embodiment, the thrombin peptide derivative comprises the following amino acid sequence: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). In another specific embodiment, the thrombin peptide derivative comprises the amino sequence of Arg-Gly-Asp-Ala-Cys-Glu-Gly-Asp-Ser-Gly-Gly-Pro-Phe-Val (SEQ ID NO:17). Alternatively, the thrombin peptide derivative comprises the amino acid sequence of SEQ ID NO:18: Asp-Asn-Met-Phe-Cys-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe. The thrombin peptide derivatives comprising the amino acid sequences SEQ ID NO: 6, 17, or 18 can optionally be amidated at the C-terminus and/or acylated at the N-terminus. Preferably, the N-terminus is free (i.e., unsubstituted) and the C-terminus is free (i.e., unsubstituted) or amidated, preferably a carboxamide (i.e., —C(O)NH₂). It is understood, however, that zero, one, two or three amino acid residues at positions 1-9 and 14-23 in the thrombin peptide derivative can differ from the corresponding amino acid in SEQ ID NO:6. It is also understood that zero, one, two or three amino acid residues at positions 1-14 and 19-33 in the thrombin peptide derivative can differ from the corresponding amino acid in SEQ ID NO:18. Preferably, the amino acid residues in the thrombin peptide derivative which differ from the corresponding amino acid in SEQ ID NO:6 or SEQ ID NO:18 are conservative substitutions, and are more preferably highly conservative substitutions. Alternatively, an N-terminal truncated fragment of the thrombin peptide derivative having at least fourteen amino acid residues or a C-terminal truncated fragment of the thrombin peptide derivative having at least eighteen amino acid residues is a thrombin peptide derivative to be used as an NPAR agonist.

A “C-terminal truncated fragment” refers to a fragment remaining after removing an amino acid or block of amino acid residues from the C-terminus. An “N-terminal truncated fragment” refers to a fragment remaining after removing an amino acid residue or block of amino acid residues from the N-terminus. It is to be understood that both C-terminal truncated fragments and N-terminal truncated fragments can optionally be amidated at the C-terminus and/or acylated at the N-terminus.

A preferred thrombin peptide derivative for use in the disclosed methods comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2). Another preferred thrombin peptide derivative for use in the disclosed method comprises the polypeptide Asp-Asn-Met-Phe-Cys-Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val-Met-Lys-Ser-Pro-Phe (SEQ ID NO:19). X₁ is Glu or Gln; X₂ is Phe, Met, Leu, His or Val. The thrombin peptide derivatives of SEQ ID NO:2 and SEQ ID NO:19 can optionally comprise a C-terminal amide and/or acylated N-terminus, as defined above. Preferably, the N-terminus is free (i.e., unsubstituted) and the C-terminus is free (i.e., unsubstituted) or amidated, preferably as a carboxamide (i.e., —C(O)NH₂). Alternatively, N-terminal truncated fragments of these preferred thrombin peptide derivatives, the N-terminal truncated fragments having at least fourteen amino acid residues, or C-terminal truncated fragments of these preferred thrombin peptide derivatives, the C-terminal truncated fragments having at least eighteen amino acid residues, can also be used in the disclosed methods.

TP508 is an example of a thrombin peptide derivative and is 23 amino acid residues long, wherein the N-terminal amino acid residue Ala is unsubstituted and the COOH of the C-terminal amino acid Val is modified to an amide represented by —C(O)NH₂ (SEQ ID NO:3). Another example of a thrombin peptide derivative comprises the amino acid sequence of SEQ ID NO:6, wherein both N- and C-termini are unsubstituted (“deamide TP508”). Other examples of thrombin peptide derivatives which can be used in the disclosed method include N-terminal truncated fragments of TP508 (or deamide TP508), the N-terminal truncated fragments having at least fourteen amino acid residues, or C-terminal truncated fragments of TP508 (or deamide TP508), the C-terminal truncated fragments having at least eighteen amino acid residues.

As used herein, a “conservative substitution” in a polypeptide is the replacement of an amino acid with another amino acid that has the same net electronic charge and approximately the same size and shape. Amino acid residues with aliphatic or substituted aliphatic amino acid side chains have approximately the same size when the total number of carbon and heteroatoms in their side chains differs by no more than about four. They have approximately the same shape when the number of branches in their side chains differs by no more than one. Amino acid residues with phenyl or substituted phenyl groups in their side chains are considered to have about the same size and shape. Listed below are five groups of amino acids. Replacing an amino acid residue in a polypeptide with another amino acid residue from the same group results in a conservative substitution:
Group I: glycine, alanine, valine, leucine, isoleucine, serine, threonine, cysteine, and non-naturally occurring amino acids with C1-C4 aliphatic or C1-C4 hydroxyl substituted aliphatic side chains (straight chained or monobranched).
Group II: glutamic acid, aspartic acid and non-naturally occurring amino acids with carboxylic acid substituted C1-C4 aliphatic side chains (unbranched or one branch point).
Group III: lysine, ornithine, arginine and non-naturally occurring amino acids with amine or guanidino substituted C1-C4 aliphatic side chains (unbranched or one branch point).
Group IV: glutamine, asparagine and non-naturally occurring amino acids with amide substituted C1-C4 aliphatic side chains (unbranched or one branch point).
Group V: phenylalanine, phenylglycine, tyrosine and tryptophan.

As used herein, a “highly conservative substitution” in a polypeptide is the replacement of an amino acid with another amino acid that has the same functional group in the side chain and nearly the same size and shape. Amino acids with aliphatic or substituted aliphatic amino acid side chains have nearly the same size when the total number of carbon and heteroatoms in their side chains differs by no more than two. They have nearly the same shape when they have the same number of branches in their side chains. Examples of highly conservative substitutions include valine for leucine, threonine for serine, aspartic acid for glutamic acid and phenylglycine for phenylalanine. Examples of substitutions which are not highly conservative include alanine for valine, alanine for serine and aspartic acid for serine.

### Modified Thrombin Peptide Derivatives

In one embodiment of the invention, the NPAR agonists are modified relative to the thrombin peptide derivatives described above, wherein cysteine residues of aforementioned thrombin peptide derivatives are replaced with amino acids having similar size and charge properties to minimize dimerization of the peptides. Examples of suitable amino acids include alanine, glycine, serine, and an S-protected cysteine. Preferably, cysteine is replaced with alanine or serine. The modified thrombin peptide derivatives have about the same biological activity as the unmodified thrombin peptide derivatives.

It will be understood that the modified thrombin peptide derivatives disclosed herein can optionally comprise C-terminal amides and/or N-terminal acyl groups, as described above. Preferably, the N-terminus of a thrombin peptide derivative is free (i.e., unsubstituted) and the C-terminus is free (i.e., unsubstituted) or amidated, preferably as a carboxamide (i. (i.e., —C(O)NH₂).

In a specific embodiment, the modified thrombin peptide derivative comprises a polypeptide Arg-Gly-Asp-Ala-Xaa-X₁-Gly-Asp-Ser-Gly-Gly-Pro-X₂-Val (SEQ ID NO:4), or a C-terminal truncated fragment thereof having at least six amino acids. More specifically, the thrombin peptide derivative comprises the polypeptide Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val (SEQ ID NO:20), or a fragment thereof comprising amino acid residues 10-18 of SEQ ID NO:20. Even more specifically, the thrombin peptide derivative comprises the polypeptide Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5), or a fragment thereof comprising amino acid residues 10-18 of SEQ ID NO:5. Xaa is alanine, glycine, serine or an S-protected cysteine. X₁ is Glu or Gln and X₂ is Phe, Met, Leu, His or Val. In one embodiment, X₁ is Glu, X₂ is Phe, and Xaa is Ala. In another embodiment, X₁ is Glu, X₂ is Phe, and Xaa is Ser. One example of a thrombin peptide derivative of this type is the polypeptide Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val (SEQ ID NO:21). A further example of a thrombin peptide derivative of this type is the polypeptide H-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-NH₂ (SEQ ID NO:22), wherein H is a hydrogen atom of alanine indicating no modification at the N-terminus, and NH₂ indicates amidation at the C-terminus as —C(O)NH₂. Zero, one, two or three amino acids in the thrombin peptide derivative differ from the amino acid at the corresponding position of SEQ ID NO:4, 20, 5, 21 or 22, provided that Xaa is alanine, glycine, serine and an S-protected cysteine. Preferably, the difference is conservative.

In another specific embodiment, the thrombin peptide derivative comprises the polypeptide Asp-Asn-Met-Phe-Xbb-Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val-Met-Lys-Ser-Pro-Phe (SEQ ID NO:23), or a fragment thereof comprising amino acids 6-28. More preferably, the thrombin peptide derivative comprises the polypeptide Asp-Asn-Met-Phe-Xbb-Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val-Met-Lys-Ser-Pro-Phe (SEQ ID NO:24), or a fragment thereof comprising amino acids 6-28. Xaa and Xbb are independently alanine, glycine, serine or an S-protected cysteine. X₁ is Glu or Gln and X₂ is Phe, Met, Leu, His or Val. Preferably X₁ is Glu, X₂ is Phe, and Xaa and Xbb are alanine. One example of a thrombin peptide derivative of this type is a polypeptide comprising the amino acid sequence Asp-Asn-Met-Phe-Ala-Gly-Pro-Phe-Val-Met-Lys-Ser-Pro-Phe (SEQ ID NO:25). A further example of a thrombin peptide derivative of this type is the polypeptide H-Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe-NH₂ (SEQ ID NO:26), wherein H is a hydrogen atom of aspartic acid indicating no modification at the N-terminus, and NH₂ indicates amidation at the C-terminus as —C(O)NH₂. Zero, one, two or three amino acids in the thrombin peptide derivative can differ from the amino acid at the corresponding position of SEQ ID NO:23, 24, 25 or 26. Xaa and Xbb are independently alanine, glycine, serine or an S-protected cysteine. Preferably, the difference is conservative.

An “S-protected cysteine” is a cysteine residue in which the reactivity of the thiol moiety, —SH, is blocked with a protecting group. Suitable protecting groups are known in the art and are disclosed, for example, in T. W. Greene and P. G. M. Wuts, Protective Groups in Organic Synthesis, 3^{rd} Edition, John Wiley & Sons, (1999), pp. 454-493, the teachings of which are incorporated herein by reference in their entirety. Suitable protecting groups should be non-toxic, stable in pharmaceutical formulations and have minimum additional functionality to maintain the activity of the thrombin peptide derivative. A free thiol can be protected as a thioether, a thioester, or can be oxidized to an unsymmetrical disulfide. Preferably the thiol is protected as a thioether. Suitable thioethers include, but are not limited to, S-alkyl thioethers (e.g., C₁-C₅ alkyl), and S-benzyl thioethers (e.g, cysteine-S-S-t-Bu). Preferably the protective group is an alkyl thioether. More preferably, the S-protected cysteine is an S-methyl cysteine.

Alternatively, the protecting group can be: 1) a cysteine or a cysteine-containing peptide (the “protecting peptide”) attached to the cysteine thiol group of the thrombin peptide derivative by a disulfide bond; or 2) an amino acid or peptide (“protecting peptide”) attached by a thioamide bond between the cysteine thiol group of the thrombin peptide derivative and a carboxylic acid in the protecting peptide (e.g., at the C-terminus or side chain of aspartic acid or glutamic acid). The protecting peptide can be physiologically inert (e.g., a polyglycine or polyalanine of no more than about fifty amino acids optionally interrupted by a cysteine), or can have a desirable biological activity.

The thrombin peptide derivatives or the modified thrombin peptide derivatives of the present invention can be mixed with a dimerization inhibitor for the preparation of a pharmaceutical composition comprising the thrombin peptide derivatives or the modified thrombin peptide derivatives of the present invention. Dimerization inhibitors can include chelating agents and/or thiol-containing compounds. An antioxidant can also be used in combination with the chelating agent and/or the thiol-containing compound.

A “chelating agent,” as used herein, is a compound having multiple sites (two, three, four or more) which can simultaneously bind to a metal ion or metal ions such as, for example, lead, cobalt, iron or copper ions. The binding sites typically comprise oxygen, nitrogen, sulfur or phosphorus. For example, salts of EDTA (ethylenediaminetetraacetic acid) can form at least four to six bonds with a metal ion or metal ions via the oxygen atoms of four acetic acid moieties (—CH₂C(O)O⁻) and the nitrogen atoms of ethylenediamine moieties (>N—CH₂—CH₂—N<) of EDTA. It is understood that a chelating agent also includes a polymer which has multiple binding sites to a metal or metal ions. Preferably, a chelating agent of the invention is non-toxic and does not cause unacceptable side effects at the dosages of pharmaceutical composition being administered in the methods of the invention. As a chelating agent of the invention, a copper-chelating agent is preferable. A “copper-chelating agent” refers to a chelating agent which can bind to a copper ion or copper ions. Examples of the copper-chelating agent include ethylenediaminetetraacetic acid (EDTA), penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine (TEPA), triethylenetetraamine and tris(2-carboxyethyl)phosphine (TCEP). Additional chelating agents are diethylenetriaminepentacetic acid (DTPA) and bathophenanthroline disulfonic acid (BPADA). EDTA is a preferred chelating agent. Typical amounts of a chelating agent present in the pharmaceutical compositions of the instant invention are in a range of between about 0.00001% and about 0.1% by weight, preferably between about 0.0001% and about 0.05% by weight.

A “pharmaceutically acceptable thiol-containing compound” as used herein is a compound which comprises at least one thiol (—SH) group and which does not cause unacceptable side effects at the dosages which are being administered. Examples of a pharmaceutically acceptable thiol-containing compound include thioglycerol, mercaptoethanol, thioglycol, thiodiglycol, cysteine, thioglucose, dithiothreitol (DTT) and dithio-bis-maleimidoethane (DIME). Typically, between about 0.001% and about 5% by weight, preferably between about 0.05% and about 1.0% by weight of a pharmaceutically acceptable thiol-containing compound is present in the pharmaceutical compositions of the invention.

An “antioxidant,” as used herein, is a compound which is used to prevent or reduce an oxidation reaction caused by an oxidizing agent such as oxygen. Examples of antioxidants include tocopherol, cystine, methionine, glutathione, tocotrienol, dimethyl glycine, betaine, butylated hydroxyanisole, butylated hydroxytoluene, vitamin E, ascorbic acid, ascorbyl palmitate, thioglycolic acid and antioxidant peptides such as, for example, turmerin. Typically, between about 0.001% and about 10% by weight, preferably between about 0.01% and about 5%, more preferably between about 0.05% and about 2.0% by weight of an antioxidant is present in the pharmaceutical compositions of the invention.

It is understood that certain chelating agents or thiol-containing compounds may also function as antioxidants, for example, tris(2-carboxyethyl)phosphine, cysteine or dithiothreitol. Other types of commonly used antioxidants, however, do not contain a thiol group. It is also understood that certain thiol-containing compounds may also function as a chelating agent, for example, dithiothreitol. Other types of commonly used chelating agents, however, do not contain a thiol group. It is also understood that the pharmaceutical compositions of the instant invention can comprise more than one chelating agent, thiol-containing compound or antioxidant. That is, for example, a chelating agent can be used either alone or in combination with one or more other suitable chelating agents.

### Thrombin Peptide Derivative Dimers

In some aspects of the present invention, the NPAR agonists of the methods are thrombin peptide derivative dimers. The dimers essentially do not revert to monomers and still have about the same biological activity as the thrombin peptide derivative monomers described above. A “thrombin peptide derivative dimer” is a molecule comprising two thrombin peptide derivatives (polypeptides) linked by a covalent bond, preferably a disulfide bond between cysteine residues. Thrombin peptide derivative dimers are typically essentially free of the corresponding monomer, e.g., greater than 95% free by weight and preferably greater than 99% free by weight. Preferably the polypeptides are the same and covalently linked through a disulfide bond.

The thrombin peptide derivative dimers of the present invention comprise the thrombin peptide derivatives described above. Specifically, thrombin peptide derivatives have fewer than about fifty amino acids, preferably about thirty-three or fewer amino acids. The thrombin peptide derivative dimers described herein are formed from polypeptides typically having at least six amino acids and preferably from about 12 to about 33 amino acid residues, and more preferably from about 12 to about 23 amino acid residues. Thrombin peptide derivative monomer subunits of the dimers have sufficient homology to the fragment of human thrombin corresponding to thrombin amino acid residues 508-530 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6)) so that NPAR is activated.

In a specific embodiment, each of the two thrombin peptide derivatives (monomers) of a dimer comprises the polypeptide Arg-Gly-Asp-Ala-Cys-X₁-Gly-Asp-Ser-Gly-Gly-Pro-X₂-Val (SEQ ID NO:1), or a C-terminal truncated fragment thereof comprising at least six amino acid residues. More specifically, a polypeptide monomer comprises the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6), or a fragment thereof comprising amino acid residues 10-18 of SEQ ID NO: 5. Even more specifically, a polypeptide monomer comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), or a fragment thereof comprising amino acid residues 10-18 of SEQ ID NO:2. X₁ is Glu or Gln and X₂ is Phe, Met, Leu, His or Val. Preferably X₁ is Glu, and X₂ is Phe. One example of a polypeptide of this type is the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:6). A further example is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3), wherein H signifies a hydrogen atom of alanine indicating no modification at the N-terminus, and NH₂ signifies amidation at the C-terminus as —C(O)NH₂. Zero, one, two or three amino acid residues in the polypeptide differ from the amino acid residue at the corresponding position of SEQ ID NO:6, 1, 2, or 3. Preferably, the difference is conservative.

One example of a thrombin peptide derivative dimer of the present invention is represented by Formula (IV):

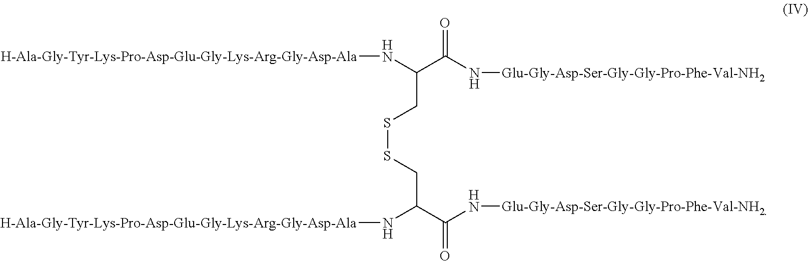

In another specific embodiment, each of the two thrombin peptide derivatives (monomers) of a dimer comprises the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr (SEQ ID NO:27), or a C-terminal truncated fragment thereof having at least twenty-three amino acid residues. More preferably, a polypeptide comprises Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val-Met-Lys-Ser-Pro-Phe-Asn-Asn-Arg-Trp-Tyr (SEQ ID NO: 8), or a C-terminal truncated fragment thereof comprising at least twenty-three amino acid residues. X₁ is Glu or Gln and X₂ is Phe, Met, Leu, His or Val. Preferably X₁ is Glu, and X₂ is Phe. One example of a polypeptide of this type is the polypeptide Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr (SEQ ID NO:27). A further example of a polypeptide of this type is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr-NH₂ (SEQ ID NO:9), wherein H signifies a hydrogen atom of alanine indicating no modification at the N-terminus, and NH₂ indicates amidation at the C-terminus —C(O)NH₂. Zero, one, two or three amino acid residues in the polypeptide differ from the amino acid residue at the corresponding position of SEQ ID NO:27, 28 or 29. Preferably, the difference is conservative.

### Methods of Treatment With NPAR Agonists

A “therapeutically effective amount” for treatment is the quantity of the NPAR agonist that results in an improvement in function, a slowing or arresting of progression, or a reduction in symptoms associated with the condition, compared to untreated or sham-treated controls. A “therapeutically effective amount” for prophylaxis is the quantity of NPAR agonist that can decrease the probability of developing the condition.

A number of tests are available to assess blood flow to an affected extremity. These tests include, for example, the ankle-brachial index test, Doppler and ultrasound (Duplex) imaging, computed tomographic angiography (CT), magnetic resonance angiography (MRA), and angiography.

The amount of the NPAR agonist administered will depend on the degree of severity of the condition, and will further depend on the release characteristics of the pharmaceutical formulation. It will also depend on the subject's health, size, weight, age, sex and tolerance to drugs. When administered more than once, the NPAR agonists can be administered at evenly spaced intervals. Each dose can be the same or different. A dose can be, for example, 0.1-500 μg, preferably 1- 50 μg of NPAR agonist, and is commonly 3, 5, 10, 30 or 50 μg.

NPAR agonists can be administered by any suitable route, including by local introduction. The NPAR agonist can be administered intravenously. The NPAR agonist can be administered to the subject in a sustained release formulation, or can be delivered by a pump or an implantable device, or by an implantable carrier comprising polymers.

Compositions comprising the NPAR agonists (e.g., peptides or peptide dimers) can be administered by any suitable route, including orally, parentally, by inhalation spray, rectally, or topically in dosage unit formulations containing conventional pharmaceutically acceptable carriers, adjuvants, and vehicles. Parental administration includes subcutaneous, intravenous, intra-arterial, intramuscular, intrastemal, intratendinous, intraspinal, intracranial, intrathoracic, intraperitoneally, by infusion techniques. Administration can be by medical instruments.

For parenteral application, particularly suitable vehicles consist of solutions, preferably oily or aqueous solutions, as well as suspensions, emulsions, or implants, including suppositories. Ampules are convenient unit dosages. It will be appreciated that the preferred amounts of active compounds used will vary according to the specific compound being utilized, the particular compositions formulated, the mode of application and the particular site of administration. Optimal administration rates for a given protocol of administration can be readily ascertained by those skilled in the art, using conventional dosage determination tests.

The NPAR agonists can be administered to the subject in conjunction with an acceptable pharmaceutical carrier as part of a pharmaceutical composition. The formulation of the pharmaceutical composition will vary according to the mode of administration selected. Suitable pharmaceutical carriers may contain inert ingredients which do not interact with the active compounds. The carriers should be biocompatible, i.e., non-toxic, non-inflammatory, non-immunogenic and devoid of other undesired reactions at the administration site. Examples of pharmaceutically acceptable carriers and other inert ingredients include, for example, saline, various buffers, alcohol, vegetable oils, polyethylene glycols, gelatin, lactose, sucrose, amylose, magnesium stearate, talc, silicic acid, viscous paraffin, perfume oil, fatty acid monoglycerides and diglycerides, petroethral fatty acid esters, hydroxymethylcellulose, polyvinylpyrrolidone, commercially available inert gels, liquids supplemented with albumin, methyl cellulose, and collagen matrix. Further examples include sterile water, physiological saline, bacteriostatic saline (saline containing about 0.9% mg/ml benzyl alcohol), phosphate-buffered saline, Hank's solution, Ringer's-lactate and the like. Standard pharmaceutical formulation techniques can be employed, such as those described in Remington's Pharmaceutical Sciences (Remington's Pharmaceutical Sciences. 21^{st} edition, Mack Publishing Company, Easton, Pa. (2005)). The pharmaceutical preparations can be sterilized and if desired, mixed with auxiliary agents, e.g., lubricants, preservatives, stabilizers, wetting agents, emulsifiers, salts for influencing osmotic pressure, buffers, colorings and the like which do not deleteriously react with the active compounds.

Pharmaceutical compositions may include gels. Gels are compositions comprising a base selected from an oleaginous base, water, or an emulsion-suspension base. To the base is added a gelling agent which forms a matrix in the base, increasing its viscosity to a semisolid consistency. Examples of gelling agents are hydroxypropyl cellulose, acrylic acid polymers, and the like. The active ingredients are added to the formulation at the desired concentration at a point preceding addition of the gelling agent or can be mixed after the gelation process.

In one embodiment, the NPAR agonists are administered in a sustained release formulation. Polymers are often used to form sustained release formulations. Examples of these polymers include poly a-hydroxy esters such as polylactic acid/polyglycolic acid homopolymers and copolymers, polyphosphazenes (PPHOS), polyanhydrides and poly (propylene fumarates).

Polylactic acid/polyglycolic acid (PLGA) homo and copolymers are well known in the art as sustained release vehicles. The rate of release can be adjusted by the skilled artisan by variation of polylactic acid to polyglycolic acid ratio and the molecular weight of the polymer (see Anderson, et al., Adv. Drug Deliv. Rev. 28:5 (1997), the entire teachings of which are incorporated herein by reference). The incorporation of poly(ethylene glycol) into the polymer as a blend to form microparticle carriers allows further alteration of the release profile of the active ingredient (see Cleek et al., J. Control Release 48:259 (1997), the entire teachings of which are incorporated herein by reference). Ceramics such as calcium phosphate and hydroxyapatite can also be incorporated into the formulation to improve mechanical qualities.

PPHOS polymers contain alternating nitrogen and phosphorous with no carbon in the polymer backbone, as shown below in Structural Formula (II):

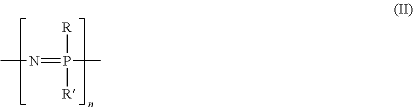

The properties of the polymer can be adjusted by suitable variation of side groups R and R′ that are bonded to the polymer backbone. For example, the degradation of and drug release by PPHOS can be controlled by varying the amount of hydrolytically unstable side groups. With greater incorporation of either imidazolyl or ethylglycol substituted PPHOS, for example, an increase in degradation rate is observed (see Laurencin et al., J. Biomed Mater. Res. 27:963 (1993), the entire teachings of which are incorporated herein by reference), thereby increasing the rate of drug release.

In certain instances it may be advantageous to co-administer one or more additional pharmacologically active agents along with an NPAR agonist. Depending on the condition, co-administration with another therapeutic agent may be appropriate, for example, an anesthetic, an analgesic, a steroid, an anti-inflammatory agent, a benzodiazepine derivative, a thrombolytic agent such as tissue plasminogen activator (tPA), or a blood thinning agent such as heparin or coumadin.

Thrombin peptide derivatives and modified thrombin peptide derivatives can be synthesized by solid phase peptide synthesis (e.g., BOC or FMOC) method, by solution phase synthesis, or by other suitable techniques including combinations of the foregoing methods. The BOC and FMOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al., Synthesis, 5: 315 (1992)). The teachings of these six articles are incorporated herein by reference in their entirety.

Thrombin peptide derivative dimers can be prepared by oxidation of the monomer (WO2004/005317). Thrombin peptide derivative dimers can be prepared by reacting the thrombin peptide derivative with an excess of oxidizing agent. A well-known suitable oxidizing agent is iodine.

A “subject” is preferably a human, but can also be an animal in need of treatment with a thrombin receptor agonist, e.g., companion animals (e.g., dogs, cats, and the like), farm animals (e.g., cows, pigs, horses and the like) and laboratory animals (e.g., rats, mice, guinea pigs and the like).

A “non-aromatic heterocyclic group” as used herein, is a non-aromatic carbocyclic ring system that has 3 to 10 atoms and includes at least one heteroatom, such as nitrogen, oxygen, or sulfur. Examples of non-aromatic heterocyclic groups include piperazinyl, piperidinyl, pyrrolidinyl, morpholinyl, thiomorpholinyl.

The term “aryl group” includes both carbocyclic and heterocyclic aromatic ring systems. Examples of aryl groups include phenyl, indolyl, furanyl and imidazolyl.

An “aliphatic group” is a straight chain, branched or cyclic non-aromatic hydrocarbon. An aliphatic group can be completely saturated or contain one or more units of unsaturation (e.g., double and/or triple bonds), but is preferably saturated, i.e., an alkyl group. Typically, a straight chained or branched aliphatic group has from 1 to about 10 carbon atoms, preferably from 1 to about 4, and a cyclic aliphatic group has from 3 to about 10 carbon atoms, preferably from 3 to about 8. Aliphatic groups include, for example, methyl, ethyl, n-propyl, iso-propyl, n-butyl, sec-butyl, tert-butyl, pentyl, cyclopentyl, hexyl, cyclohexyl, octyl and cyclooctyl.

Suitable substituents for an aliphatic group, an aryl group or a non-aromatic heterocyclic group are those which do not significantly lower therapeutic activity of the NPAR agonist, for example, those found on naturally occurring amino acids. Examples include —OH, a halogen (—Br, —Cl, —I and —F), —O(Rₑ), —O—CO—(Rₑ), —CN, —NO₂, —COOH, ═O, —NH₂—NH(Rₑ), —N(Rₑ)², —COO(Rₑ), —CONH₂, —CONH(Rₑ),—CON(Rₑ)₂, —SH, —S(Rₑ), an aliphatic group, an aryl group and a non-aromatic heterocyclic group. Each Rₑ is independently an alkyl group or an aryl group. A substituted aliphatic group can have more than one substituent.

### EXAMPLES

### Example 1

### Materials and Methods

### Endothelial Cell Culture

Human coronary artery endothelial cells (HCAE cells, Lonza, Walkersville, Md.) were cultured in 5% CO₂ at 37° C. in endothelial cell growth medium (EGM) supplemented with 2% fetal bovine serum and Single Quot supplements (Clonetics, San Diego, Calif.; containing epidermal growth factor, hydrocortisone, vascular endothelial growth factor, fibroblast growth factor, insulin growth factor, ascorbic acid, heparin, gentamycin, and amphotericin B). Cells were used between passages 4 and 6 for these studies. HCAE cells were plated at a density of 50,000 cells per well in 12-well plates and were grown for 3 days to reach confluency. Two-day post-confluent cells were treated with TNFα or TP508. Cells were then incubated in normoxic or 1% hypoxic conditions for the indicated times. In some experiments, cells were pretreated with TP508 for 1 h before TNFα stimulation. For RNA extraction, HCAE cells were cultured in 6-well plates.

### RT-PCR

Total RNA was isolated using an Ambion isolation kit as described by the manufacturer.

RT-PCR for eNOS was performed by Ready-to-Go RT-PCR Beads (Amersham Biosciences) using a two-step protocol. First, cDNA synthesis was achieved by incubating 1 μg total RNA with 1 μg random hexamers (pdN6) as primers and M.-MuLV reverse transcriptase at 42° C. for 60 min. Then, after heating at 95° C. for 5 min, PCR was carried out performing 25 or 30 cycles of denaturation (95° C. for 30 s), annealing (62° C. for 30 s), and extension (72° C. for 30 s), ending with a single final extension at 72° C. for 7 min. The sequences for human eNOS primers were as follows: sense 5′-GCA CCG GCA TCA CCA GGA AGA AGA-3′ (SEQ ID NO:35) and antisense 5′-CCG CCG CCA AGA GGA CAC CAG T-3′ (SEQ ID NO:36) (Sitges, M. et al., Int. J. Cardiol. 105(1):74-79, 2005). 18S primers (Ambion) were used to amplify 18S ribosomal RNA as an internal normalization control. PCR-amplified products were visualized by 1% agarose gel electrophoresis.

### SYBR Green Real-Time PCR

Real time quantitative PCR was used to determine relative expression of eNOS mRNA in endothelial cells treated with TP508 or TNF. Samples were coded to provide blinded analysis. One μg of total RNA was reverse-transcribed using Taqman Reverse Transcription Reagents Kit (ABI) as specified by the manufacturer. Quantitative PCR amplifications were done using 2 μl of cDNA in a total volume of 25 μl using SYBR green probes in the SYBR Green PCR Master Mix (ABI). All PCR assays were run in the ABI Prism 7000 Sequence Detection System. The SYBR green PCR primers were as follows: human eNOS sense: 5′-GCG GCT GCA TGA CAT TGA G-3′ (SEQ ID NO:37), antisense: 5′-GTC GCG GTA GAG ATG GTC AAG T-3′ (SEQ ID NO:38). The reverse-transcribed cDNA sample cycle threshold values were determined from triplicate samples and normalized to the 18S “housekeeping” gene.

### Western Blot Analysis

The cell lysates were subjected to SDS-PAGE in a 10% polyacrylamide gel and transferred to a nitrocellulose membrane (0.2-μm, Invitrogen). After blocking with 5% milk, the membrane was incubated overnight at 4° C. with primary antibodies against phospho-eNOS (Ser 1177) and eNOS (Cell Signaling Technology, Beverly, Mass.), human type-1 arginase (clone 9C5) (Cell Sciences, Canton, Mass.), or GAPDH (Santa Cruz Biotechnology, Santa Cruz, Calif.). HRP-conjugated anti-mouse or anti-rabbit antibodies (Cell Signaling Technology) were used as secondary antibodies (HRP, horseradish peroxidase). The immunoblots were developed using Immobilon Western Detection Reagents (Millipore Corporation).

### Example 2

### TP508 Blocks TNFα Induced Upregulation of Arginase 1

As described above, ED and loss of NO dependent signaling can arise either from a decrease in NOS activity or by an increase in arginase activity that depletes cellular levels of L-arginine. In both hypoxia and inflammation, it has been reported that the level of TNFα is elevated and is thought to contribute to ED by affecting one or both of these NO related activities. To evaluate the potential effects of TP508 on ED, early passage human coronary artery endothelial cells (HCAE cells) were cultured under normoxic conditions and were treated with TNFα alone or with the combination of TNFα and TP508.

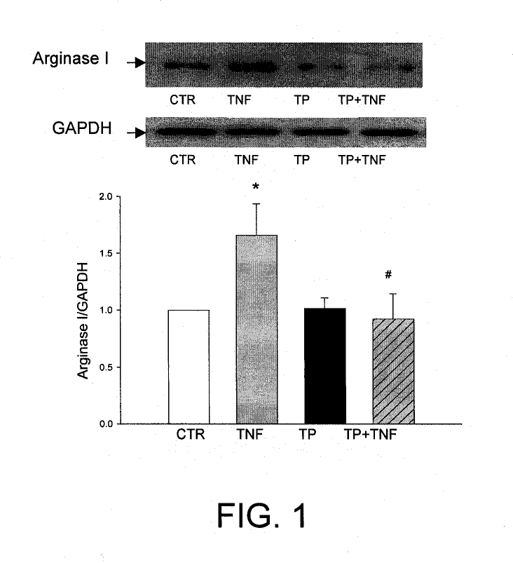

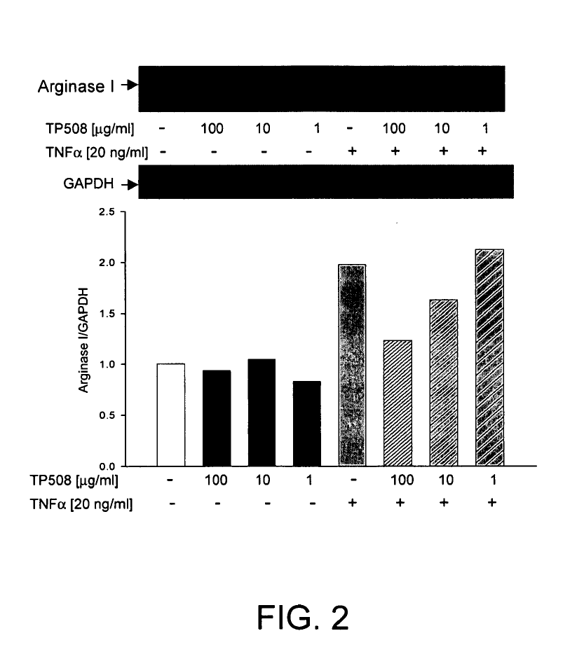

Western blots of HCAE cell lysates, using antibody specific for human type-1 arginase showed that TNFα treatment caused a significant increase in the expression of arginase 1 (ARG1) relative to control cultures (FIG. 1). As shown in FIGS. 1 and 2,

TP508 treatment alone had no effect on ARG1 expression, but completely blocked the TNFα-induced increase in ARG1 expression. The TP508 inhibition of TNFα stimulated ARG 1 expression is dose dependent with half maximal inhibition occurring at a TP508 concentration of 10 μg/ml (FIG. 2). Therefore, TP508 appears to block TNFα-induced signals that lead to an increase in ARG1. This inhibitory effect of TP508 appears to be specific for a subset of TNFα effects, since TNFα stimulated phosphorylation of Erk1/2 and p70 S6 kinase were not inhibited by TP508.

### Example 3

### TP508 Stimulates eNOS Expression and eNOS Phosphorylation

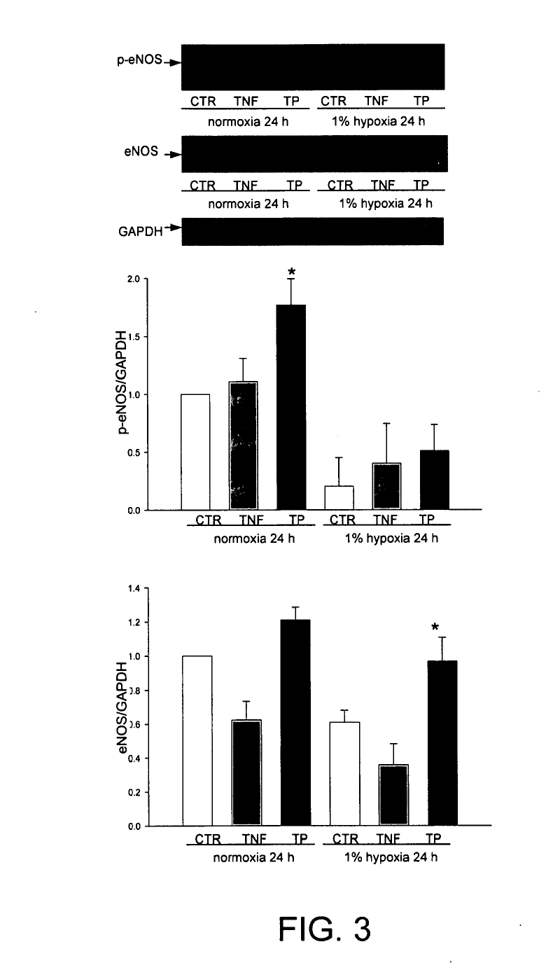

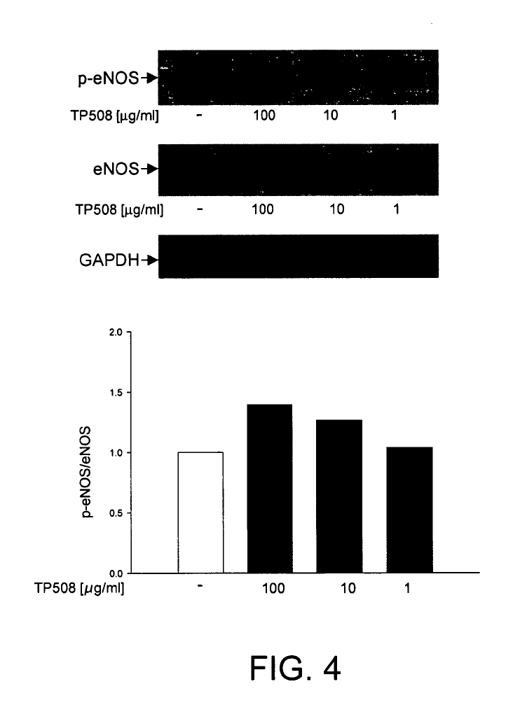

Western blots of HCAE cell lysates, using antibody specific for eNOS phosphorylated at S1177 showed that TNFα treatment, or exposure of cells to hypoxic conditions (1% O₂) for 24 h, reduced the expression of eNOS by 45%, relative to normoxic controls (FIG. 3). As shown, TP508 prevented the decreased expression of eNOS caused by hypoxia to retain eNOS expression levels at levels similar to those seen in cells cultured under normoxic conditions. In contrast, TP508 addition together with TNFα was not able to inhibit the TNFα induced decrease in eNOS expression (not shown). Under normoxic and hypoxic conditions, TP508 increased eNOS phosphorylation relative to normoxic and hypoxic control cultures 1.8-fold and 2.5-fold, respectively (FIG. 3). Although some of this phosphorylation may be due to increased expression of eNOS, the increased phosphorylation cannot be explained by increased expression alone. Additional experiments examining the effect of TP508 on eNOS expression and phosphorylation in cells cultured under normoxic conditions confirmed that TP508 stimulated eNOS phosphorylation and showed that this effect is dose dependent with half maximal response at ˜10 μg/ml (FIG. 4).

### Example 4

### TP508 Upregulates eNOS mRNA

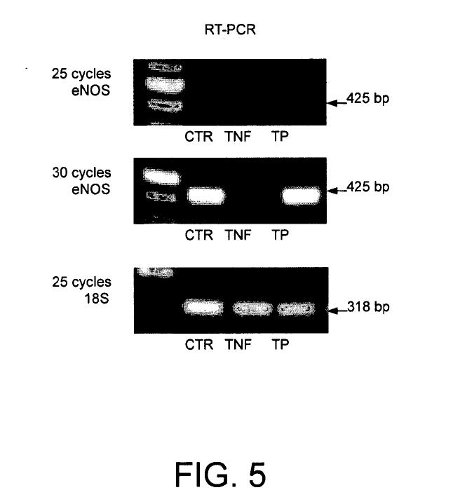

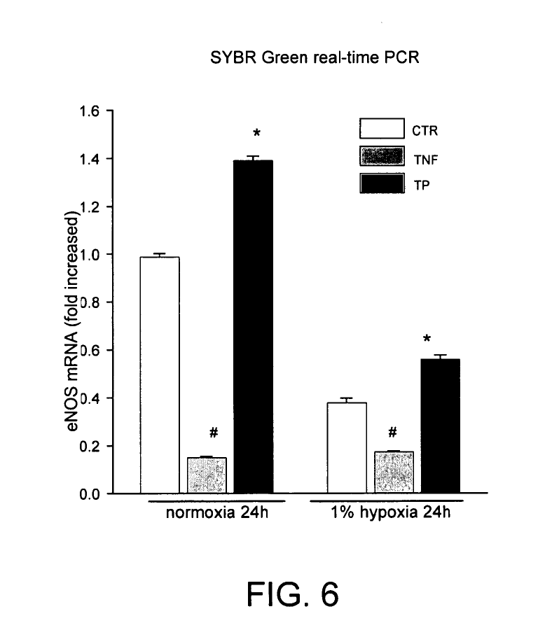

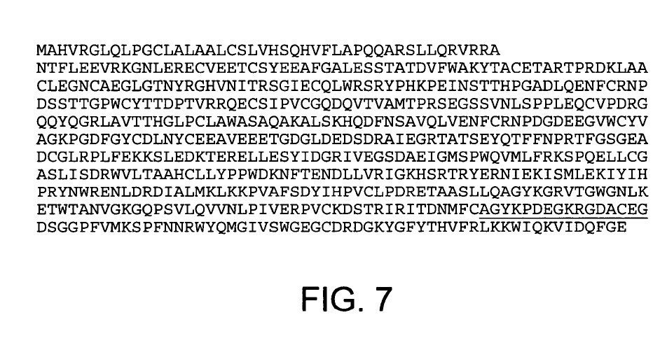

Consistent with the protein expression data, RT-PCR analysis of mRNA from HCAE cells showed that TP508 upregulates eNOS mRNA expression (FIG. 5). This effect of TP508 is shown at 25 cycles of RT-PCR where an eNOS mRNA band is seen in TP508-treated cells, but not in control cells. In contrast, TNFα treatment drastically decreased expression relative to controls as demonstrated after 30 cycles of RT-PCR. To quantify this effect of TP508, we used SYBR Green real-time PCR analysis of reverse transcribed mRNA samples. As shown in FIG. 6, after 24 h of culture under normoxic conditions, TP508 increased the level of eNOS mRNA by 40% relative to controls (p<0.05). In contrast, TNFα decreased the eNOS mRNA level by ˜80%. A 24-hour exposure of the cells to hypoxia (1% oxygen) also decreased the control levels of eNOS mRNA by ˜60%. TP508 treatment of these cells partially prevented the hypoxia induced decrease. As shown, TP508 treated hypoxic cells had eNOS mRNA levels ˜40% over those of hypoxic control cells (p<0.05).

### Example 5

### TP508 Potentiates the VEGF-Induced Activation of eNOS

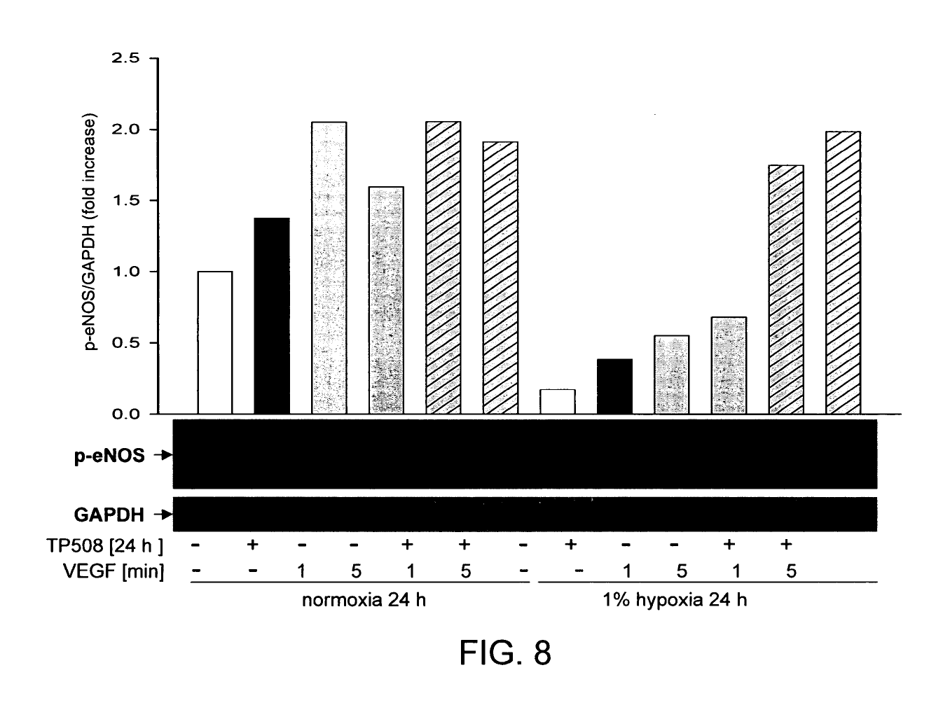

Human coronary artery endothelial (HCAE) cells (Lonza, Walkersville, Md.) were cultured in the presence or absence of TP508 [50 μg/ml] in normoxic and hypoxic [1% O₂] conditions for 24 h and then stimulated with the angiogenic growth factor, human VEGF [50 ng/ml] for 1 or 5 min. Human VEGF-induced eNOS activation was determined by Western blotting using an antibody recognizing the activated form of eNOS (phosphorylated at S1177) (Cell Signaling, Danvers, Mass.). The membrane was re-probed with anti-GAPDH (glyceraldehyde-3-phosphate dehydrogenase) antibody to show equal protein loading. A bar graph representing densitometric analysis of the activated eNOS Western blot after different treatments is shown in FIG. 8.

As shown in FIG. 8, in normoxic cells, human VEGF induces transient phosphorylation of eNOS on serine 1177 to activate the enzyme which is maximum at 1 minute (2-fold) and has declined after 5 minutes stimulation. If cells were pretreated with TP508 prior to human VEGF stimulation, the phosphorylation of eNOS was prolonged and remained near maximum stimulation for 5 minutes. Thus, TP508 potentiates the ability of human VEGF to signal eNOS phosphorylation by extending the period of maximal stimulation.

In hypoxic cells (cultured in 1% O₂ for 24 hours), the level of human VEGF-stimulated eNOS phosphorylation is decreased ˜4 fold at 1 min treatment compared to normoxic cells. Thus, hypoxia significantly reduces human VEGF-stimulated activation of eNOS. However, hypoxic cells pretreated with TP508 showed human VEGF-induced activation of eNOS at levels equivalent to that seen in normoxic cells. Thus, TP508 treatment of hypoxic cells restores the ability of human VEGF to stimulate eNOS activation to the level observed in normoxic cells.

### Example 6

### TP508 Enhances Endothelial Cell Migration Towards VEGF

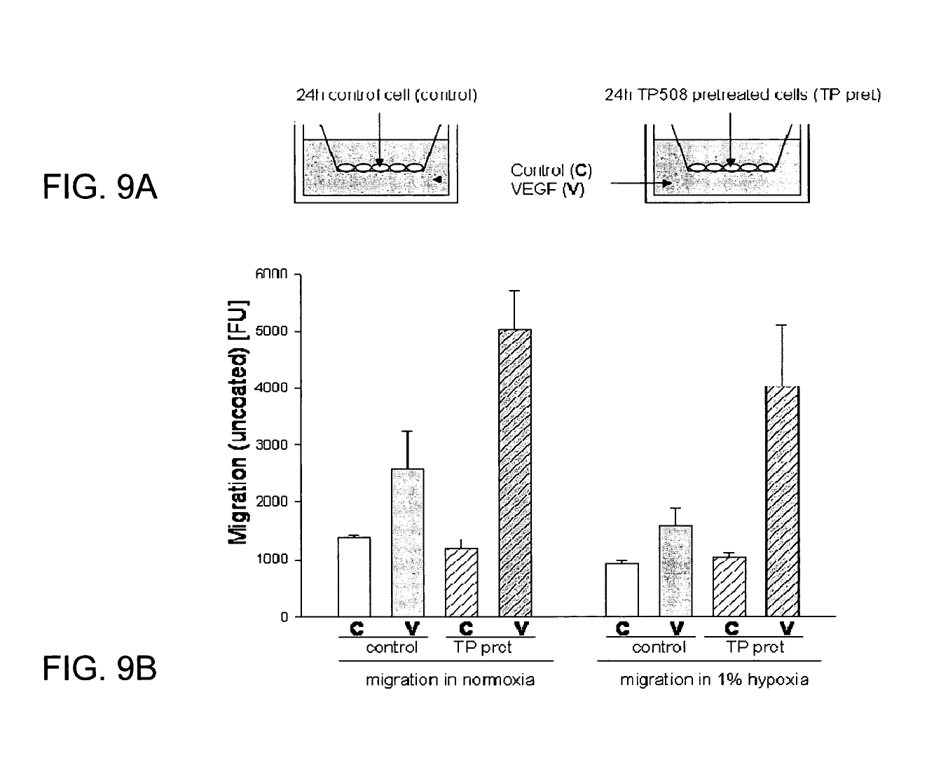

The ability of a test substance to attract endothelial cells and stimulate their migration through pores in the membrane is one of several tests to determine the angiogenic potential of test substances. FIG. 9A shows the design of experiments to measure migration of endothelial cells toward a chemoattractant. Prior to migration assay, cells were cultured with or without TP508 to determine the effect of TP508 on endothelial migration.

Human coronary artery endothelial (HCAE) cells (Lanza Walkersville, Inc., Walkersville, Md.) were cultured in the absence (control) or presence of TP508 [50 μg/ml] (“TP pret” in FIG. 9A and FIG. 9B) for 24 hours. Transmembrane cell migration assays were performed using BD FluoroBlok inserts (BD Bioscience, Bedford, Mass.) as described by the vendor. Control or TP508 pretreated cells were added into the top of the inserts. Human VEGF [10 ng/ml] (V) or medium alone (C) was added to the lower chamber of the insert plate as a chemoattractant. Endothelial migration was performed in normoxic or 1% hypoxic conditions. After a 22-hour incubation, cells were labeled post-migration with Calcein AM and measured by detecting the fluorescence of the cells that migrated to the underside of the insert membrane.

FIG. 9B shows the effect of TP508 treatment on migration of endothelial cells toward the angiogenic factor human VEGF (human recombinant VEGF-A 165, R&D System, Minneapolis, Minn.).

The results show that human VEGF stimulates normal control endothelial cell migration by ˜2 fold relative to media control cells when assayed in normoxic conditions (180%) and slightly less (˜150%) under hypoxic conditions relative to media control cells. Endothelial cells that were pretreated with TP508 showed cell migration toward human VEGF ˜5-fold and ˜4 fold relative to media controls when cells were assayed under normoxic and hypoxic conditions, respectively. TP508 pretreatment, thus, enhances endothelial migration toward human VEGF 2- to 3-fold relative to untreated control cells. Since this cell migration assay is one measure of the angiogenic potential of cells, these results demonstrate that TP508 treatment more than doubles the angiogenic potential of human VEGF for endothelial cells under normoxic conditions as well as under hypoxic conditions where angiogenic responses to human VEGF are diminished.

### Example 7

### TP508 Increases Angiogenic Response of Endothelial Cells Toward Human VEGF

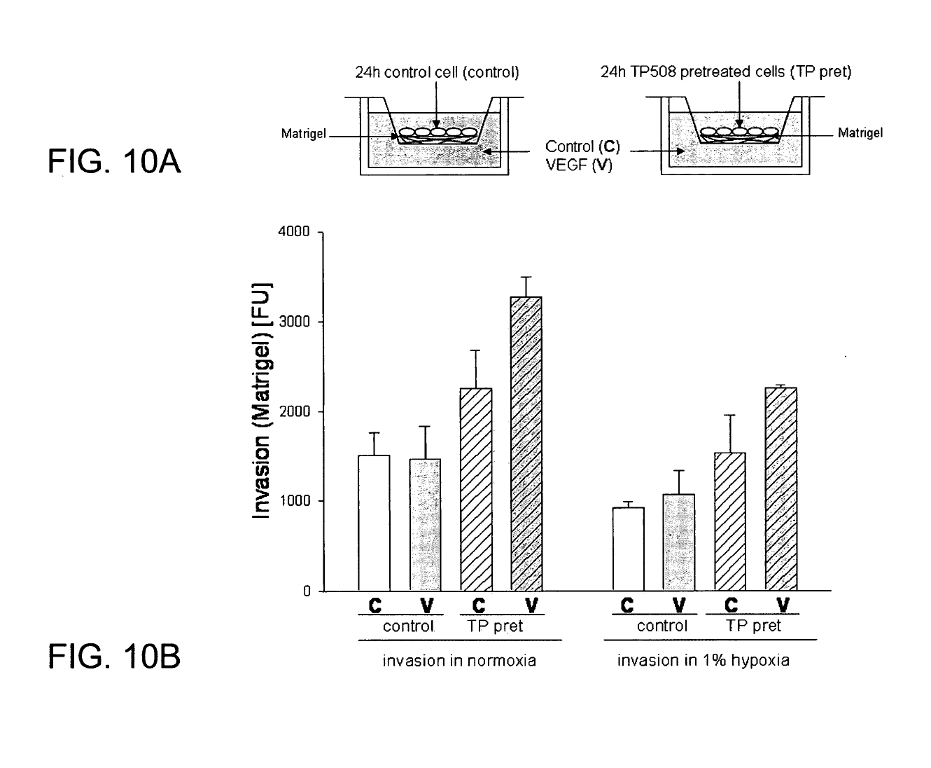

Invasion of endothelial cells through a Matrigel matrix is one of many assays used to determine the angiogenic potential of test substances and is thought to be more predictive of angiogenesis in vivo than a simple chemotactic assay through open membrane pores since the cells must degrade and invade the matrix to move into and through the pores in the membrane. FIG. 10A shows the design of experiments to measure invasion of endothelial cells through Matrigel toward a chemoattractant.

Human coronary artery endothelial (HCAE) cells (Lonza Walkersville, Inc., Walkersville, Md.) were cultured in the absence (control) or presence of TP508 [50 μg/ml] (TP pret) for 24 hours. Endothelial cell invasion assays were performed using BD BioCoat™ Angiogenesis System (BD Bioscience, Bedford, Mass.) which utilizes FluoroBlok inserts coated with BD Matrigel Matrix (BD Bioscience, Bedford, Mass.). Control or TP508 pretreated cells were added into the top of the inserts. Medium containing human VEGF [10 ng/ml human recombinant VEGF-A 165aa, R&D System, Minneapolis, Minn.] (V) or medium alone (C) was added to the lower chamber of the insert plate as a chemoattractant to determine angiogenic response to human VEGF. Endothelial cell invasion was performed in normoxic or hypoxic (1% O₂) conditions. After 22 hours of incubation, cells were labeled post-invasion with Calcein AM and measured by detecting the fluorescence of the cells that migrated to the underside of insert membrane.

FIG. 10B shows the effect of TP508 treatment on invasion of endothelial cells toward human VEGF. The results show that control endothelial cells assayed in normoxic conditions or under hypoxic conditions are not stimulated by human VEGF to degrade Matrigel and migrate through the membrane toward human VEGF. In contrast, endothelial cells that were pre-incubated with TP508 show increased invasive properties over control cells that were not pretreated with TP508. In addition, these cells now respond to human VEGF (-50% more invasion than observed in TP508 pretreated cells without human VEGF and nearly twice as much invasion as control cells toward VEGF). These results demonstrate that TP508 treatment increases the ability of endothelial cells to respond angiogenically to human VEGF under conditions where non-TP508 treated control cells do not respond at all to human VEGF treatment.

### Example 8

### Effects of TP508 on VEGF mRNA Expression in Normoxic and Hypoxic HCMVE Cells

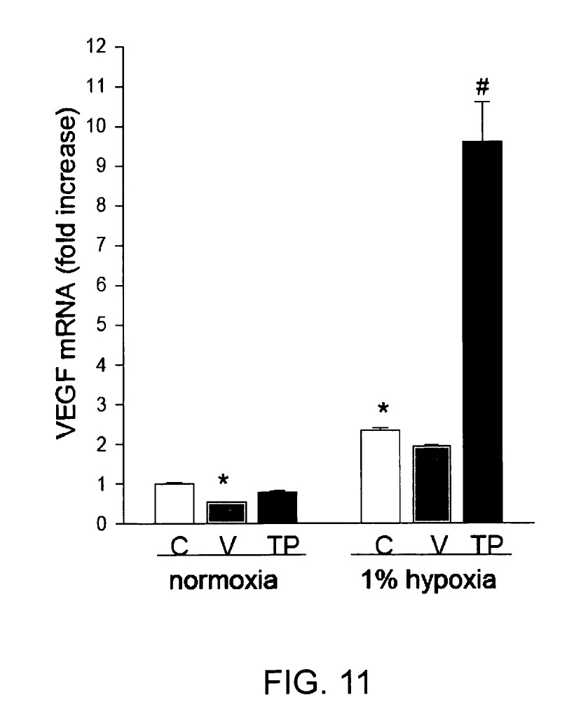

Human cardiac microvascular endothelial (HCMVE) cells were treated with VEGF (10 ng/ml) (V) or TP508 (50 μg/ml) (TP) and cultured in normoxic or 1% hypoxic conditions for 24 h. Real-time PCR analyses show changes in steady-state levels of VEGF mRNA after treatment. Data from one experiment performed in triplicate are shown in FIG. 11 as mean±SD. *p<0.01 compared to control (C) cells in normoxia. ^{#}p<0.01 compared to control (C) cells in hypoxia.

Real-time PCR analyses showed that VEGF stimulation decreased VEGF mRNA expression (2-fold) compared to control cell in normoxic condition. As expected, hypoxia increased VEGF mRNA (2.4-fold) compared to normoxic control cells. TP508 treatment had no effect on steady-state level of VEGF mRNA in normoxic cells. However, hypoxic cells treated with TP508 expressed ˜4-fold and ˜10-fold higher levels of VEGF mRNA expression compared to control hypoxic or control normoxic cells, respectively (FIG. 11). In contrast, VEGF stimulation had no effect, or decreased VEGF mRNA in hypoxic cells. By this experiment, TP508 enhances the effect of hypoxia to up-regulate VEGF expression.

### Example 9

### Effect of TP508 Pre-Treatment on Carbachol Induced Relaxation

Rat aortic rings (endothelial cell layer intact) were prepared. Rings were treated with no TP508 (control) or 1 mM TP508 for 1 hour. Rings were contracted with 500 nM norepinephrine, followed by increasing doses of carbachol (0, 1, 10, 100, 500, 750, 1000 and 5000 nM). Mean values±SEM are shown; n=2 animals.

Norepinephrine contracted rings, with intact endothelium, relax in response to increasing doses of carbachol (Furchgott, R. F. and Zawadzki, J. V., Nature 288: 373-376(1980)). If TP508 is a smooth muscle relaxant, TP508 treatment prior to a norepinephrine-carbachol dosing regimen should result in an increase in carbachol induced relaxation compared to the control, i.e., a shift of the carbachol dose response curve to the left relative to the control curve.

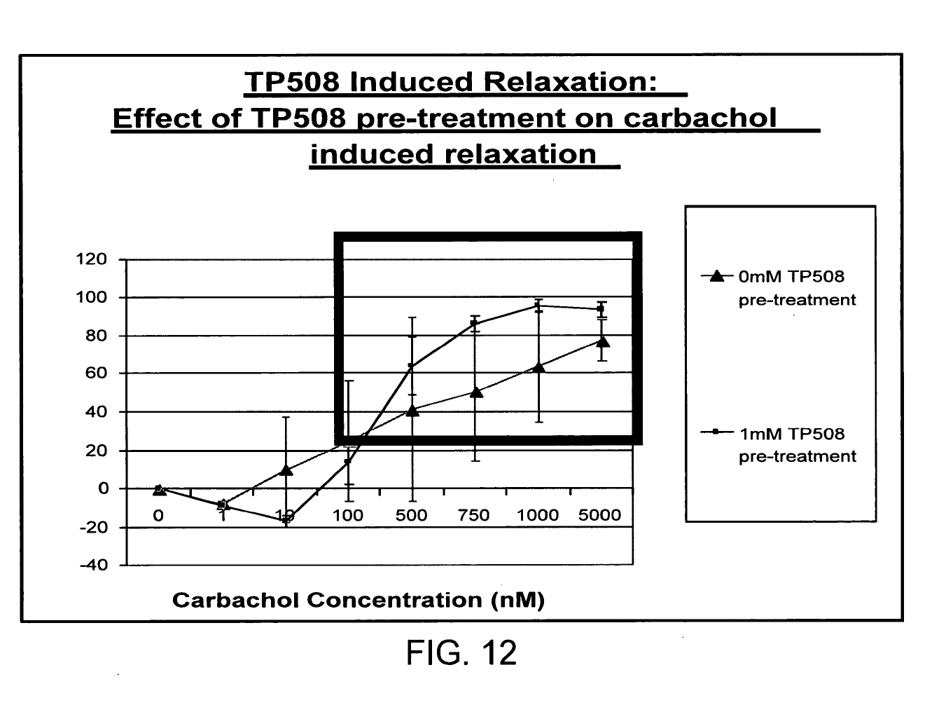

FIG. 12 demonstrates that at increased carbachol concentrations (greater than 250 nM; rectangular box) TP508 pre-treated rings showed increased relaxation relative to controls. Furthermore, TP508 pre-treatment leads to a sigmoidal carbachol dose response curve. The control rings produced a linear carbachol dose response curve.

### Example 10

### Studies on Human Lung Microvascular Endothelial Cells

### HLME (Human Lung Microvascular Endothelial Cells; Alternatively, HLMVE Cells)

HLME cells were tested with VEGF, TP508 and serum-deprivation. Both lines observed showed increased survival when treated with both VEGF and TP508. VEGF alone increased survival at lower levels, and TP508 alone had little effect.

VEGF, eNOS, VEGFR1 and VEGFR2 were observed responding to TP508 treatment under hypoxic and normoxic conditions. When HLME cells were studied under normoxic conditions, only eNOS was positively regulated by TP508 treatment. When HLME cells were studied under hypoxic conditions, all four of these genes were upregulated at the mRNA level over the hypoxic untreated control.

TABLE
HLME SUMMARY

| Treatment | Responses | |
|---|---|---|
| Cells were deprived of | Treating with | Treating with VEGF and |
| serum, treated with | TP508 alone did | TP508 increased survival |
| VEGF and TP508 | not increase cell | beyond VEGF alone. |
| | survival rate | |
| Cells treated with | eNOS expression | VEGF, VEGFR1 and |
| TP508: normoxic | was up regulated | VEGFR2 expression: no |
| | | effect |
| Cell treated with | eNOS expression | VEGF, VEGFR1 and |
| TP508: hypoxic | was up regulated | VEGFR2 expression were |
| | | upregulated |

### Example 11

### Effects on Endothelial Function/Dysfunction in Isolated Coronary Arterioles

Coronary microvessels (arterioles) were isolated at day 60 from hearts of adult Yucatan swine that had undergone chronic occlusion of the left circumflex coronary artery (LCX) using an ameroid constrictor, and from nonischemic left anterior descending arteries (LAD) as described. Subepicardial arteriolar branches (60-100 μm internal diameter and 1 to 1.5 mm in length without branches, in situ) were carefully dissected from the LAD or LCX. Each arteriole was cannulated with glass micropipettes and pressurized to 60 cm H₂O intraluminal pressure without flow. Internal diameters of the vessel were measured throughout the experiment using video microscopic techniques incorporated with MacLab (ADInstruments, Inc) data acquisition system. Following the development of tone (spontaneous constriction to about 70% of maximal diameter), we determined the concentration to diameter relationship for endothelium-dependent NO-mediated vasodilators adenosine (0.1 nM to 10 μM) and serotonin (0.1 nM to 1 μM), ATP sensitive potassium channel opener pinacidil (0.1 μM to 3 μM), and endothelium-independent vasodilator sodium nitroprusside (1 nM to 0.1 mM). The vasodilatory responses to these agonists were established before and after extraluminal incubation of arterioles. At the end of the experiments, each vessel was relaxed with 100 μM sodium nitroprusside in a physiologically balanced calcium-free salt solution containing ethylenediaminetetraacetic acid (EDTA, 1 mM) to obtain its maximal diameter at 60 cm H₂O intraluminal pressure. All diameter changes in response to agonists were normalized to this dilation and expressed as a percentage of maximal dilation.

To determine if chronic ischemia in this model of LCX occlusion created coronary artery endothelial dysfunction and if TP508 treatment affected NO signaling, coronary arterioles were isolated from nonischemic branches of left anterior descending arteries (LAD) and from occluded, ischemic, LCX of TP508- and placebo-treated hearts. The isolated arterioles were then treated ex vivo with adenosine, serotonin, sodium nitroprusside, or pinacidil. If arterioles have endothelial dysfunction with impaired NO production, they should show diminished vasodilation in response to adenosine and serotonin (which are endothelium-dependent NO-mediated vasodilators), but not to sodium nitroprusside and pinacidil, which relax smooth muscle cells independent of endothelial cell response by donating NO to activate cyclic GMP and opening smooth muscle cell ATP-sensitive potassium channels, respectively.

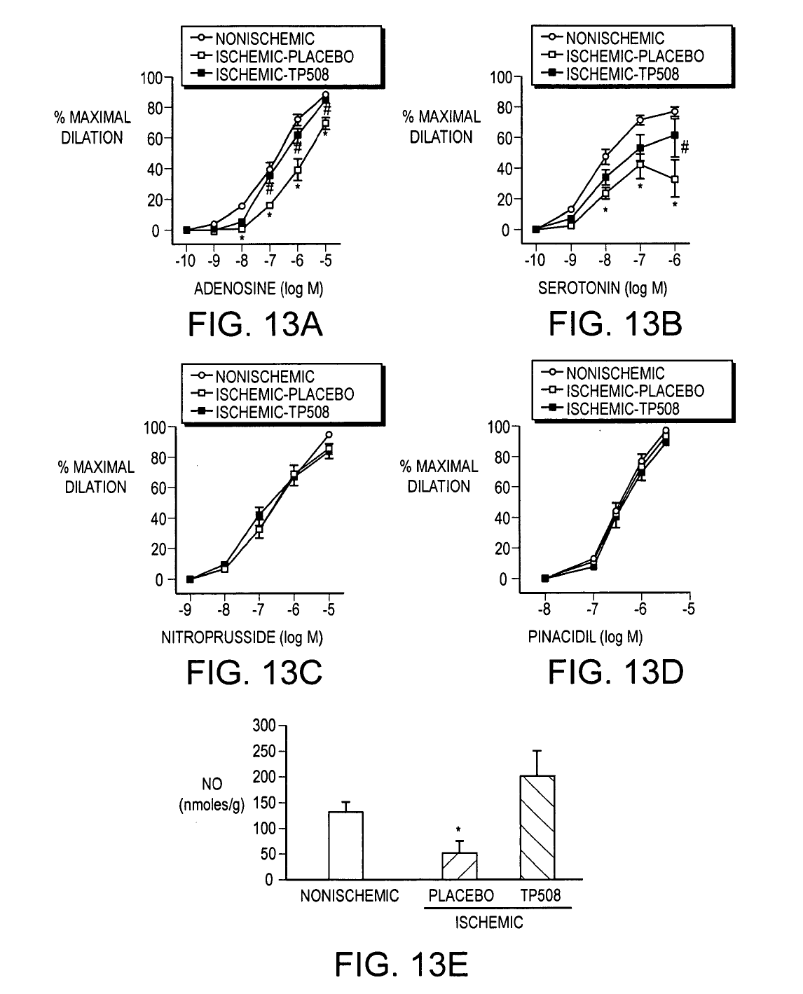

Ischemic arterioles show significantly diminished dilation responses to adenosine or serotonin relative to nonischemic vessels (FIGS. 13A and 13B). As shown, ischemia causes a rightward shift in the dose-response curve with ischemic arterioles requiring an order of magnitude more drug to achieve the same degree of dilation as nonischemic arterioles. This effect of hypoxia was not seen when isolated vessels were exposed to nitroprusside or pinacidil (FIG. 13C and 13D). Thus, hypoxic arterioles dilate normally in response to NO delivered via NO donor and to a smooth muscle relaxing drug, but demonstrate classic endothelial dysfunction with diminished ability of endothelial cells to produce NO in response to serotonin and adenosine.

TP508 significantly increased arteriolar response to adenosine and serotonin relative to the responses of arterioles isolated from ischemic placebo-treated hearts (FIGS. 13A and 13B). Response to adenosine, in fact, appears to be restored in arterioles isolated from ischemic TP508-treated hearts to the level seen in nonischemic control arterioles (FIG. 13A).

To confirm that this TP508 effect on vasodilation represented a reversal of endothelial dysfunction in these arterioles, we determined the effect of hypoxia and TP508 on the ability of these arterioles to generate NO and the levels of eNOS expression. As shown in FIG. 13E, NO production by isolated arterioles from the ischemic LCX of placebo-treated hearts was decreased relative to nonischemic control arterioles from the LAD. Arterioles from ischemic LCX of TP508-treated hearts, however, had levels of NO production that exceeded the nonischemic control level. PCR analysis of mRNA from a limited number of these arterioles showed that eNOS mRNA expression was decreased in arterioles from the ischemic LCX relative to nonischemic control arterioles. Similar to its effect on NO production, TP508 treatment restored eNOS mRNA expression in ischemic arterioles to a level similar to or greater than that found in nonischemic arterioles. These results suggest that TP508 reverses endothelial dysfunction in these arterioles by a process that includes the upregulation of eNOS allowing more NO production.

### Example 12

### Oxidative Stress Responses of Human Umbilical Vein Endothelial Cells

Human umbilical vein endothelial cells (HUVECs)were seeded into 12-well plates pre-coated with cell attachment factor at a density of 50,000 cells per well. Cells were allowed to attach overnight, at which point the plating medium was changed to basal medium (EBM), lacking growth factors and antibiotics, but supplemented with insulin-transferrin-selenium (Gibco, Grand Island, N.Y.) to prevent the cells from lifting off the plates during incubation in serum-free medium. After 24 hours, one set of wells was pretreated with 1 ml 10 μg/ml TP508 in EBM for 20 min at 37°. These wells were subsequently treated with 350 μl of 10 μg/ml TP508+100 mM H₂O₂. The other wells were treated with 350 μl/well of EBM alone, 100 mM H₂O₂, or 10 μg/ml TP508. The treated cells were incubated at 37° for 1 hr, at which point the media was removed and microfuged for 2 mM at 6,000 rpm at room temperature. Duplicate 100 μl aliquots of each supernatant were transferred to flat-bottom 96-well plates, and subjected to the lactate dehydrogenase cytotoxicity detection assay (Boehringer Mannheim, Indianapolis, Ind.). The assay quantifies the activity of lactate dehydrogenase (LDH), released by damaged cells into the culture medium. The results were expressed as the % of cytotoxicity as given by the formula:

% cytotoxicity=(sample activity−low control activity/high control activity−low control activity)*100%.

Low control=LDH activity of cells in EBM alone.

High control=LDH activity of cells lysed by 1% Triton X-100 in EBM.

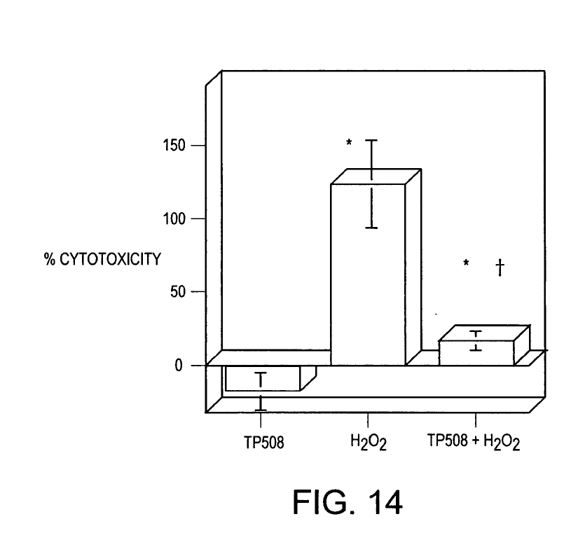

The results in FIG. 14 suggest that TP508 pretreatment produced rapid changes that protected the cells from the cytotoxic effects of hydrogen peroxide. TP508 may activate glutathione peroxidase, catalase, or NADPH production, which would help glutathione peroxidase convert H₂O₂ to water.

### Example 13

### Apoptosis Studies in B11-C Mouse Fibroblasts

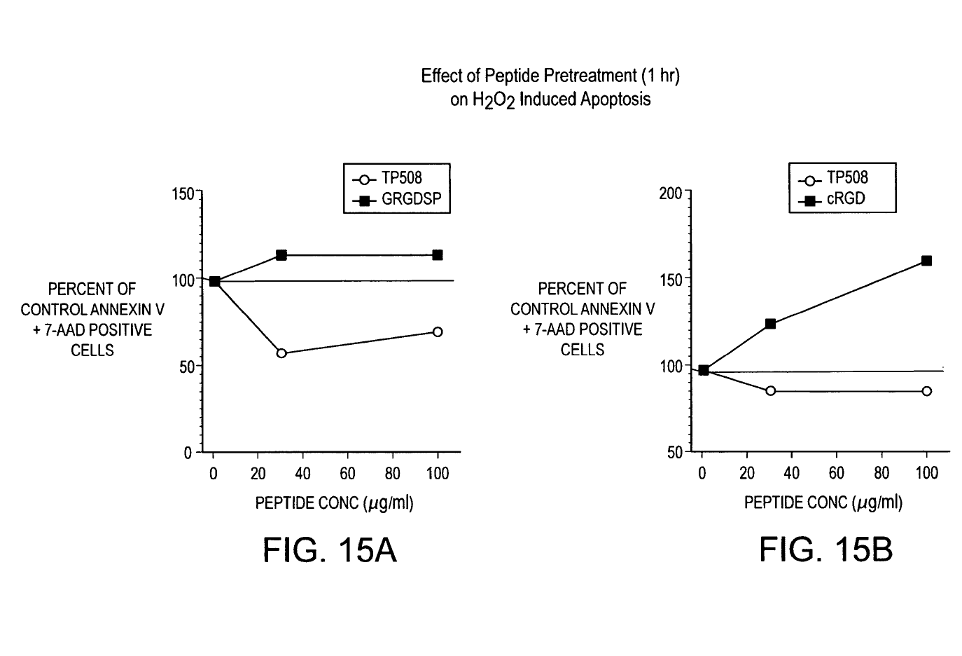

Cells were cultured in serum-free medium for 48 hr, treated with indicated concentrations of TP508 or integrin activating RGD peptides, and then treated for 24 hours with 1.2 mM hydrogen peroxide. Apoptosis was determined by Annexin V and 7-AAD staining using flow microfluorimetry. TP508 has a protective effect, decreasing the number of Annexin V and 7-AAD positive cells, while integrin activating peptides appear to promote apoptosis. See FIGS. 15A and 15B.

### Example 14

### Gene Array Analysis

A number of gene array analyses have been done using human microvascular endothelial cells (HMVECs). These data have been examined to see if particular mediators of apoptosis were altered by TP508.

Adult and neonatal human microvascular endothelial cells (HMVECs) obtained from Cambrex were plated into flasks coated with cell attachment factor in serum containing medium with growth factor supplements, switched to serum-free medium and cultured in normoxic or hypoxic (1% O₂) environments. Twenty-four hours before harvesting, the cells were treated with TP508 or medium alone. Total RNA was extracted and processed for expression analysis using Affymetrics chips.

Heat maps from different sets of these cells all show significant differences in up-regulated genes and down-regulated genes between control and cells treated for 24 hours with TP508. A number of these genes are involved in cell cycle regulation, cell proliferation, and cell death. The reports also show potential signaling pathways that may be involved or utilized by TP508.

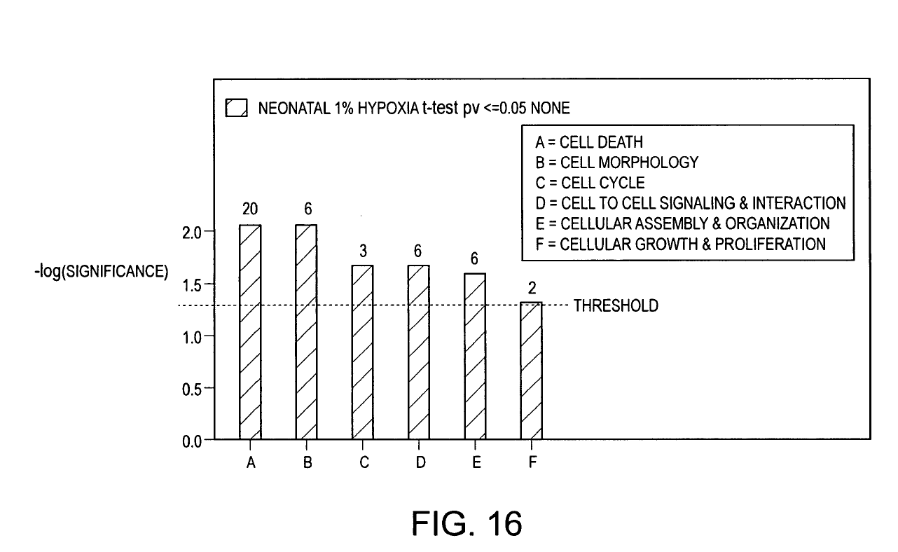

In neonatal HMVECs, TP508 produced effects on stress related pathways that are high in hypoxic cells, but reduced due to TP508 treatment. TP508 significantly changed expression of 20 genes associated with cell death and others associated with cell morphology, cell cycle, and cell signaling. See FIG. 16.

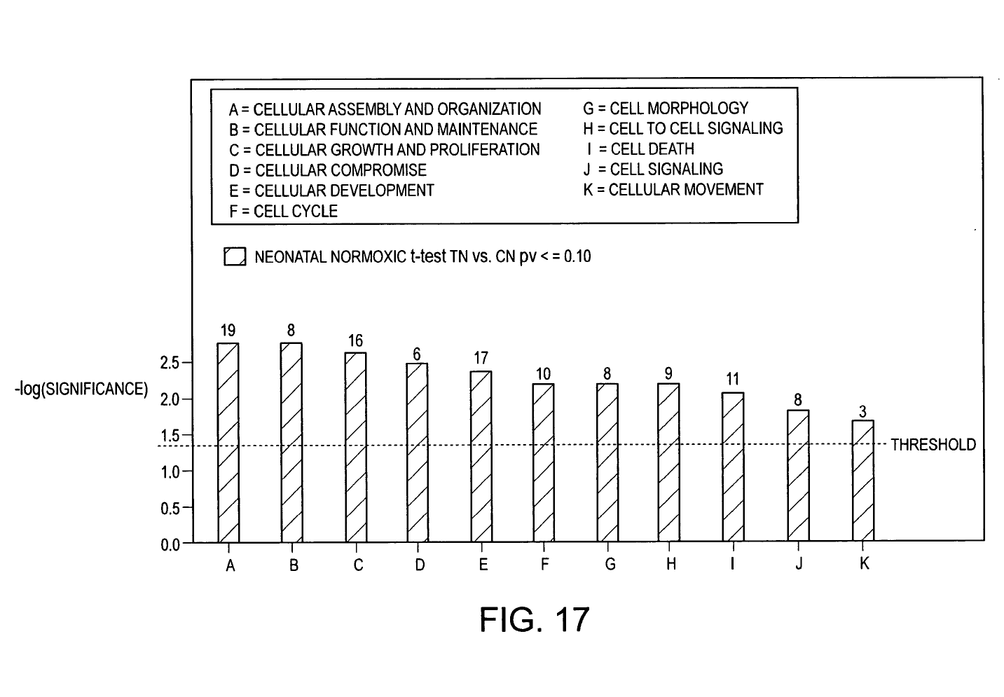

Several different signaling pathways appear to be activated by TP508. The signal pathways with the highest potential significance based on this limited gene set (4 and 5 genes out of 82 for each pathway) were EGF, IL4, IL2, VEGF, FGF, and IGF. Among genes affecting developmental processes and function, the most genes (11 and 12) are involved in hematological and skeletal/muscular systems. Cellular process genes suggest effects on cell organization, maintenance, growth/proliferation, development, cell cycle, cell morphology, cell signaling, cell death, and movement. See FIG. 17.

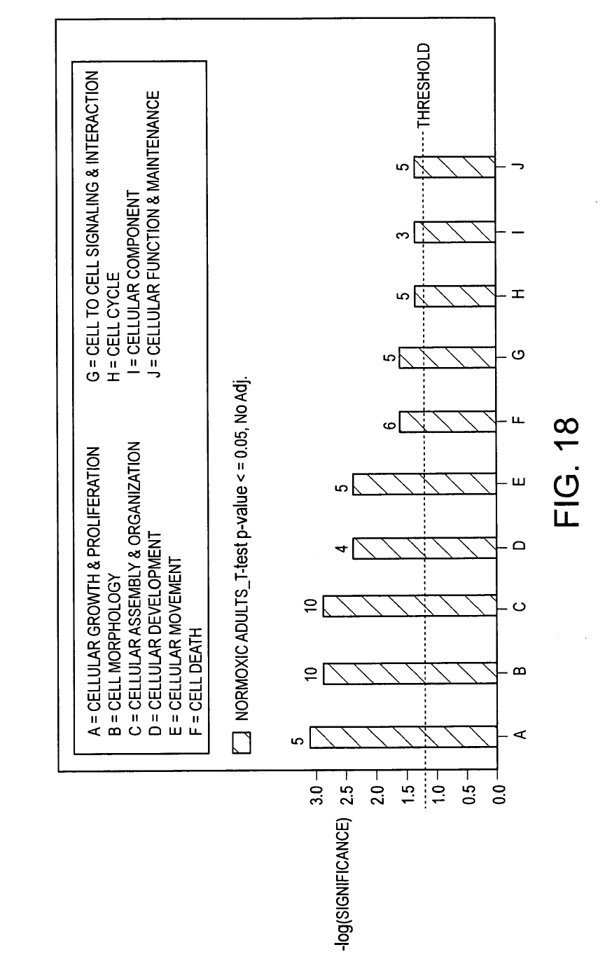

In adult HMVECs, the most significant cellular processes affected by TP508 were growth/proliferation, cell assembly, cell movement, cell signaling, and cell components. See FIG. 18. A number of genes that significantly changed were related to injury. Significant gene changes were noted that related to PPAR signaling and estrogen receptor signaling, but not to signaling through insulin receptors, ERK/MAPk signaling, or integrin signaling. The capsase-1 dominant-negative inhibitor pseudo-ICE appears to be down-regulated by hypoxia, but significantly up-regulated by TP508 treatment of these hypoxic cells. CRCR1 or DDC that induces apoptosis (Fume, C. et al., Proc Natl Acad Sci USA 103:4128-4133, 2006), is up-regulated by hypoxia, but down-regulated or returned to normal levels when hypoxic cells are treated with TP508.

Another example of a gene with a pattern similar to DDC is the PH domain and leucine rich repeat protein phosphatase (PHLPP) that de-phosphorylates AKT. AKT is phosphorylated by a number of survival factors and is a key regulator for preventing apoptosis. By up-regulation or activation of PHLPP, AKT is dephosphorylated to induce apoptosis (Gao, T. et al., Mol Cell. 18(1):13-24, 2005). In HMVECs, hypoxia up-regulates PHLPP, but TP508 treatment of hypoxic cells down-regulates the expression of this phosphatase.

### Example 15

### Apoptosis Genes in HCAE Cells

Gene array data has been collected using human coronary artery endothelial (HCAE) cells. These studies have focused on the effects of TNFα and the ability of TP508 to prevent or reverse the effects of TNFα. TNFα is known to induce apoptosis in a number of cell types including endothelial cells.

These gene array data in HCAE cells have been analyzed for genes that cluster to a specific profile. In this case, we only looked at genes that were up-regulated by TNFα more than 3-fold, but were not significantly up-regulated by TP508 or TNFα when these cells are treated with TP508. In the set of data studied, over a thousand genes fall into this cluster profile and appear to be up-regulated by more than 3-fold by TNFα treatment alone, but are not upregulated by TNFα if the cells are pretreated with TP508. Of these, 35 gene sets representing more than 30 genes are specifically involved in induction of apoptosis.

While this invention has been particularly shown and described with references to example embodiments thereof, it will be understood by those skilled in the art that various changes in form and details may be made therein without departing from the scope of the invention encompassed by the appended claims.

1. A method for treating peripheral arterial disease in a subject in need thereof, the method comprising administering to the subject a therapeutically effective amount of an agonist of a non-proteolytically activated thrombin receptor.

2. The method of claim 1, wherein the peripheral arterial disease is occlusive.

3. The method of claim 2, wherein the peripheral arterial disease is acute peripheral arterial occlusion.

4. The method of claim 2, wherein the disease is Buerger's disease.

5. The method of claim 1, wherein the peripheral arterial disease is characterized by intermittent claudication.

6. The method of claim 1, wherein the peripheral arterial disease is functional.

7-8. (canceled)

9. The method of claim 1, wherein the agonist is a thrombin peptide derivative comprising the amino acid sequence Asp-Ala-R, wherein R is a serine esterase conserved sequence, and the thrombin peptide derivative comprises from about 12 to about 23 amino acid residues.

10-16. (canceled)

17. The method of claim 9, wherein the thrombin peptide derivative comprises an N-terminus which is unsubstituted, and a C-terminus which is unsubstituted or a C-terminal amide represented by —C(O)NH₂.

18. (canceled)

19. The method of claim 17, wherein the thrombin peptide derivative comprises the polypeptide Arg-Gly-Asp-Ala-Cys-X₁-Gly-Asp-Ser-Gly-Gly-Pro-X₂-Val (SEQ ID NO:1), wherein X₁ is Glu or Gln and X₂ is Phe, Met, Leu, His or Val.

20-22. (canceled)

23. The method of claim 17, wherein the thrombin peptide derivative comprises the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), an N-terminal truncated fragment of the thrombin peptide derivative having at least fourteen amino acid residues, or a C-terminal truncated fragment of the thrombin peptide derivative having at least eighteen amino acid residues, wherein X₁ is Glu or Gln and X₂ is Phe, Met, Leu, His or Val.

24. The method of claim 1, wherein the agonist is the polypeptide H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:3).

25-32. (canceled)

33. The method of claim 17, wherein the thrombin peptide derivative comprises the polypeptide Ala-Gly-Tyr-Lys-Pro-Asp-Glu-Gly-Lys-Arg-Gly-Asp-Ala-Xaa-X₁-Gly-Asp-Ser-Gly-Gly-Pro-X₂-Val (SEQ ID NO:5) or a fragment thereof comprising amino acids 10-18 of SEQ ID NO:5, wherein Xaa is alanine, glycine, serine or an S-protected cysteine; X₁ is Glu or Gln; and X₂ is Phe, Met, Leu, His or Val.

34-38. (canceled)

39. The method of claim 1 any of claim 1 6 or 58 60, wherein the agonist is a peptide dimer comprising two thrombin peptide derivatives 12 to 23 amino acid residues in length which, independently, comprise the polypeptide Asp-Ala-Cys-X₁-Gly-Asp-Ser-Gly-Gly-Pro-X₂-Val (SEQ ID NO:10), wherein X₁ is Glu or Gln and X₂ is Phe, Met, Leu, His or Val, or a C-terminal truncated fragment thereof having at least six amino acid residues, provided that zero, one, two, or three amino acid residues in the polypeptide differ from those residues in the corresponding position of SEQ ID NO:10; said thrombin peptide derivatives optionally comprising a C-terminal amide; and said thrombin peptide derivatives optionally comprising an acylated N-terminus; the dimer is essentially free of monomer;
the thrombin peptide derivatives are the same;
the thrombin peptide derivatives are covalently linked through a disulfide bond; and
the thrombin peptide derivatives consist of from about 12 to about 23 amino acid residues.

40-44. (canceled)

45. The method of claim 39, wherein the thrombin peptide derivatives each comprise an N-terminus which is unsubstituted; and a C-terminus which is unsubstituted or a C-terminal amide represented by —C(O)NH₂.

46-49. (canceled)

50. The method of claim 45, wherein the thrombin peptide derivatives comprise the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), wherein X₁ is Glu or Gln and X₂ is Phe, Met, Leu, His or Val or a fragment thereof comprising amino acid residues 10-18 of SEQ ID NO:2.

51. The method of claim 45, wherein the thrombin peptide derivatives comprise the polypeptide Ala-Gly-Tyr-Lys-Pro-Asp-Glu-Gly-Lys-Arg-Gly-Asp-Ala-Cys-X₁-Gly-Asp-Ser-Gly-Gly-Pro-X₂-Val (SEQ ID NO:2), wherein X₁ is Glu or Gln and X₂ is Phe, Met, Leu, His or Val.

52-55. (canceled)

56. The method of claim 1, wherein the agonist is a peptide dimer (SEQ ID NO: 3) represented by the following structural formula:

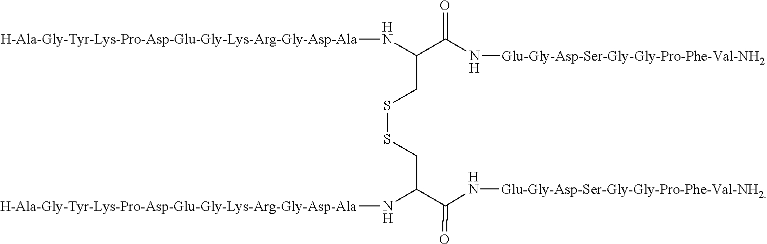

57. (canceled)

58. A method for treating hemorrhagic stroke in a subject in need thereof, the method comprising administering to the subject a therapeutically effective amount of an agonist of a non-proteolytically activated thrombin receptor.

59. A method for treating ischemic stroke in a subject in need thereof, the method comprising administering to the subject a therapeutically effective amount of an agonist of a non-proteolytically activated thrombin receptor.

60. A method for treating deep vein thrombosis in a subject in need thereof, the method comprising administering to the subject a therapeutically effective amount of an agonist of a non-proteolytically activated thrombin receptor.


SEQUENCE LISTING (40 sequences)

SEQ ID NO: 1 - protein, 14 aa, Artificial Sequence
  Description of Artificial Sequence: Synthetic peptide
  MOD_RES (6)..(6): Glu or Gln
  MOD_RES (13)..(13): Phe, Met, Leu, His, or Val
RGDACXGDSGGPXV

SEQ ID NO: 2 - protein, 23 aa, Artificial Sequence
  Description of Artificial Sequence: Synthetic peptide
  MOD_RES (15)..(15): Glu or Gln
  MOD_RES (22)..(22): Phe, Met, Leu, His, or Val
  see specification as filed for detailed description of substitutions and preferred embodiments
AGYKPDEGKRGDACXGDSGGPXV

SEQ ID NO: 3 - protein, 23 aa, Artificial Sequence
  Description of Artificial Sequence: Synthetic peptide
  N-term H
  C-term NH2
AGYKPDEGKRGDACEGDSGGPFV

SEQ ID NO: 4 - protein, 14 aa, Artificial Sequence
  Description of Artificial Sequence: Synthetic peptide
  MOD_RES (5)..(5): Ala, Gly, Ser, or S-protected Cys
  MOD_RES (6)..(6): Glu or Gln
  MOD_RES (13)..(13): Phe, Met, Leu, His, or Val
RGDAXXGDSGGPXV

SEQ ID NO: 5 - protein, 23 aa, Artificial Sequence
  Description of Artificial Sequence: Synthetic peptide
  MOD_RES (14)..(14): Ala, Gly, Ser, or S-protected Cys
  MOD_RES (15)..(15): Glu or Gln
  MOD_RES (22)..(22): Phe, Met, Leu, His, or Val
AGYKPDEGKRGDAXXGDSGGPXV

SEQ ID NO: 6 - protein, 23 aa, Artificial Sequence
  Description of Artificial Sequence: Synthetic peptide
AGYKPDEGKRGDACEGDSGGPFV

SEQ ID NO: 7 - protein, 10 aa, Artificial Sequence
  Description of Artificial Sequence: Synthetic peptide
EGKRGDACEG

SEQ ID NO: 8 - protein, 33 aa, Artificial Sequence
  Description of Artificial Sequence: Synthetic polypeptide
  MOD_RES (15)..(15): Glu or Gln
  MOD_RES (22)..(22): Phe, Met, Leu, His, or Val
  see specification as filed for detailed description of substitutions and preferred embodiments
AGYKPDEGKRGDACXGDSGGPXVMKSPFNNRWY

SEQ ID NO: 9 - protein, 33 aa, Artificial Sequence
  Description of Artificial Sequence: Synthetic polypeptide
  N-term H
  C-term NH2
AGYKPDEGKRGDACEGDSGGPFVMKSPFNNRWY

SEQ ID NO: 10 - protein, 12 aa, Artificial Sequence
  Description of Artificial Sequence: Synthetic peptide
  MOD_RES (4)..(4): Glu or Gln
  MOD_RES (11)..(11): Phe, Met, Leu, His, or Val
DACXGDSGGPXV

SEQ ID NO: 11 - protein, 14 aa, Artificial Sequence
  Description of Artificial Sequence: Synthetic peptide
  MOD_RES (5)..(5): Ala, Gly, Ser, or S-protected Cys
RGDAXEGDSGGPFV

SEQ ID NO: 12 - protein, 23 aa, Artificial Sequence
  Description of Artificial Sequence: Synthetic peptide
  N-term H
  MOD_RES (15)..(15): Glu or Gln
  MOD_RES (22)..(22): Phe, Met, Leu, His or Val
  C-term NH2
AGYKPDEGKRGDACXGDSGGPXV

SEQ ID NO: 13 - protein, 12 aa, Artificial Sequence
  Description of Artificial Sequence: Synthetic peptide
  MOD_RES (2)..(2): Ala or Ser
  MOD_RES (4)..(4): Glu or Gln
  MOD_RES (11)..(11): Phe, Met, Leu, His, or Val
DXCXGDSGGPXV

SEQ ID NO: 14 - protein, 10 aa, Artificial Sequence
  Description of Artificial Sequence: Synthetic peptide
CEGDSGGPFV

SEQ ID NO: 15 - protein, 10 aa, Artificial Sequence
  Description of Artificial Sequence: Synthetic peptide
  MOD_RES (2)..(2): Glu or Gln
  MOD_RES (9)..(9): Phe, Met, Leu, His, or Val
CXGDSGGPXV

SEQ ID NO: 16 - protein, 4 aa, Artificial Sequence
  Description of Artificial Sequence: Synthetic peptide
RGDA

SEQ ID NO: 17 - protein, 14 aa, Artificial Sequence
  Description of Artificial Sequence: Synthetic peptide
RGDACEGDSGGPFV

SEQ ID NO: 18 - protein, 33 aa, Artificial Sequence
  Description of Artificial Sequence: Synthetic polypeptide
DNMFCAGYKPDEGKRGDACEGDSGGPFVMKSPF

SEQ ID NO: 19 - protein, 33 aa, Artificial Sequence
  Description of Artificial Sequence: Synthetic polypeptide
  MOD_RES (20)..(20): Glu or Gln
  MOD_RES (27)..(27): Phe, Met, Leu, His, or Val
DNMFCAGYKPDEGKRGDACXGDSGGPXVMKSPF

SEQ ID NO: 20 - protein, 23 aa, Artificial Sequence
  Description of Artificial Sequence: Synthetic peptide
  MOD_RES (14)..(14): Ala, Gly, Ser, or S-protected Cys
AGYKPDEGKRGDAXEGDSGGPFV

SEQ ID NO: 21 - protein, 23 aa, Artificial Sequence
  Description of Artificial Sequence: Synthetic peptide
AGYKPDEGKRGDAAEGDSGGPFV

SEQ ID NO: 22 - protein, 23 aa, Artificial Sequence
  Description of Artificial Sequence: Synthetic peptide
  N-term H
  C-term NH2
AGYKPDEGKRGDAAEGDSGGPFV

SEQ ID NO: 23 - protein, 33 aa, Artificial Sequence
  Description of Artificial Sequence: Synthetic polypeptide
  MOD_RES (5)..(5): Ala, Gly, Ser, or S-protected Cys
  MOD_RES (19)..(19): Ala, Gly, Ser, or S-protected Cys
  see specification as filed for detailed description of substitutions and preferred embodiments
DNMFXAGYKPDEGKRGDAXEGDSGGPFVMKSPF

SEQ ID NO: 24 - protein, 33 aa, Artificial Sequence
  Description of Artificial Sequence: Synthetic polypeptide
  MOD_RES (5)..(5): Ala, Gly, Ser, or S-protected Cys
  MOD_RES (19)..(19): Ala, Gly, Ser, or S-protected Cys
  MOD_RES (20)..(20): Glu or Gln
  MOD_RES (27)..(27): Phe, Met, Leu, His or Val
  see specification as filed for detailed description of substitutions and preferred embodiments
DNMFXAGYKPDEGKRGDAXXGDSGGPXVMKSPF

SEQ ID NO: 25 - protein, 33 aa, Artificial Sequence
  Description of Artificial Sequence: Synthetic polypeptide
DNMFAAGYKPDEGKRGDAAEGDSGGPFVMKSPF

SEQ ID NO: 26 - protein, 33 aa, Artificial Sequence; identical to SEQ ID NO: 25
  Description of Artificial Sequence: Synthetic polypeptide
  N-term H
  C-term NH2

SEQ ID NO: 27 - protein, 33 aa, Artificial Sequence; identical to SEQ ID NO: 9
  Description of Artificial Sequence: Synthetic polypeptide

SEQ ID NO: 28 - protein, 23 aa, Artificial Sequence
  Description of Artificial Sequence: Synthetic peptide
  N-term H
  C-term NH2
AGYKPDEGKRGDASEGDSGGPFV

SEQ ID NO: 29-34 - skipped (6 entries)

SEQ ID NO: 35 - DNA, 24 bp, Artificial Sequence
  Description of Artificial Sequence: Synthetic primer
gcaccggcatcaccaggaagaaga

SEQ ID NO: 36 - DNA, 22 bp, Artificial Sequence
  Description of Artificial Sequence: Synthetic primer
ccgccgccaagaggacaccagt

SEQ ID NO: 37 - DNA, 19 bp, Artificial Sequence
  Description of Artificial Sequence: Synthetic primer
gcggctgcatgacattgag

SEQ ID NO: 38 - DNA, 22 bp, Artificial Sequence
  Description of Artificial Sequence: Synthetic primer
gtcgcggtagagatggtcaagt

SEQ ID NO: 39 - protein, 622 aa, Homo sapiens [sequence omitted]

SEQ ID NO: 40 - protein, 6 aa, Artificial Sequence
  Description of Artificial Sequence: Synthetic peptide
GRGDSP